(19) United States
(12) Patent Application Publication
(10) Pub. No.: US 2026/0001844 A1
(43) Pub. Date: Jan. 1, 2026

(54) COMPOUNDS AND METHODS FOR THE TREATMENT AND PREVENTION OF HIV

(71) Applicants: THE TRUSTEES OF THE UNIVERSITY OF PENNSYLVANIA, Philadelphia, PA (US); HOSPITALIER DE L'UNIVERSITÉ DE MONTRÉAL, Montreal (CA); THE HENRY M. JACKSON FOUNDATION FOR THE ADVANCEMENT OF MILITARY MEDICINE, INC., Bethesda, MD (US)

(72) Inventors: Amos B. SMITH, Merion Station, PA (US); Andrés FINZI, Montreal (CA); Shilei DING, Montreal (CA); Tyler HIGGINS, Philadelphia, PA (US); Xuchen ZHAO, Beijing (CN); Daniel LEE, Philadelphia, PA (US); Marzena PAZGIER, Bethesda, MD (US); Dung N. NGUYEN, Bethesda, MD (US); William D. TOLBERT, Bethesda, MD (US)

(21) Appl. No.: 18/867,605

(22) PCT Filed: May 23, 2023
(86) PCT No.: PCT/US2023/067367

§ 371 Date: Nov. 20, 2024

Related U.S. Application Data
(60) Provisional application No. 63/345,294, filed on May 24, 2022.
(60) Provisional application No. 63/391,049, filed on Jul. 21, 2022.

(51) Int. Cl. C07D 211/60; A61K 31/454; A61K 31/496; A61K 45/06; A61P 31/18; C07D 401/06

(52) U.S. Cl. CPC C07D 211/60; A61K 31/454; A61K 31/496; A61K 45/06; A61P 31/18; C07D 401/06

(57) ABSTRACT

Compositions and methods for treating HIV using compounds that sensitize HIV-1 infected cells to antibody-dependent cellular cytotoxicity.

### CROSS REFERENCE TO RELATED APPLICATIONS

The present application claims priority to and the benefit of U.S. patent application nos. 63/345,294, filed May 24, 2022; and 63/391,049, filed Jul. 21, 2022; both entitled “Compounds And Methods For The Treatment And Prevention Of HIV”. All foregoing applications are incorporated herein by reference in their entireties for any and all purposes.

### GOVERNMENT RIGHTS

This invention was made with government support under GM056550 and AI150471 awarded by the National Institutes of Health. The government has certain rights in the invention.

### TECHNICAL FIELD

The disclosure pertains to compounds and methods for sensitizing HIV-1 infected cells to antibody-dependent cellular cytotoxicity.

### BACKGROUND

With approximately 37 million people living with HIV worldwide and an estimated 2 million new infections reported each year, the need to derive novel strategies aimed at eradicating HIV-1 infection remains a critical worldwide challenge. One potential strategy involves eliminating infected cells via antibody-dependent cellular cytotoxicity (ADCC). HIV-1 has evolved sophisticated mechanisms to conceal epitopes located in its envelope glycoprotein (Env) that are recognized by ADCC-mediating antibodies present in sera from HIV-1 infected individuals. Compounds that expose relevant anti-Env epitopes and sensitize HIV-1 infected cells to ADCC may be useful in the treatment of HIV. The gp120 molecule of HIV-1 is a glycoprotein that is part of the outer layer of the virus which facilitates HIV entry into the host cell.

Small molecule CD4 (T-cells) mimetic compounds (CD4mc) might have an advantage over soluble CD4 (sCD4) to modulate Env conformation at the surface of cells infected with primary viruses, unmasking CD4i epitopes that are recognized by antibodies present in HIV+ sera. More small molecule CD4 are needed.

### SUMMARY

The disclosure is directed to compounds of formula II and stereoisomers of formula II, as well as pharmaceutically acceptable salts thereof.

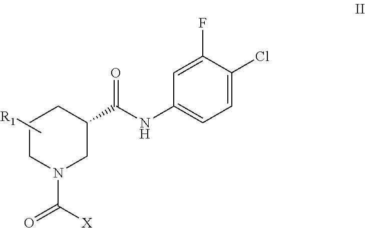

The disclosure is directed to compounds of formula I and stereoisomers of formula I, as well as pharmaceutically acceptable salts thereof.

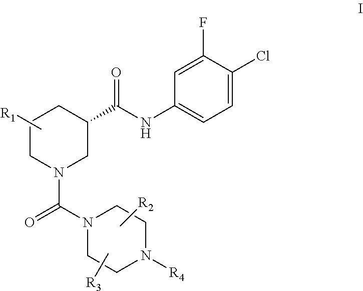

Methods of making and using the compounds of formula I are also described.

Methods of making and using the compounds of formula II are also described.

### BRIEF DESCRIPTION OF THE DRAWINGS

The foregoing summary, as well as the following detailed description, will be better understood when read in conjunction with the appended drawings, which show exemplary embodiments for the purposes of illustration.

FIG. 1. To evaluate the direct virus-neutralizing ability of the compounds, MLV (A) or HIV-1CH_{58TF} virus was incubated with indicated amounts of different compounds or DMSO, then the mixture was added to TZM-bl cells. Luciferase activity was measured.

FIG. 2. Primary CD4 T cells isolated from PBMC were infected with HIV-1_{CH58TF} for 48 hours. For cell surface staining (A), 1:1000 diluted of HIV+ plasma were used in the presence of the different tested compounds (50 μM or with an equivalent volume of vehicle (DMSO). For ADCC (B), the assay determines susceptibility to ADCC mediated by 1/1,000 dilution of plasma from HIV-1-infected individuals in the presence of different compounds (50 μM) or DMSO.

FIG. 3. Depicts Crystal structures of MCG-IV-210 CD4mc derivatives in complex with a gp120 core. (A) Blow-up views into the gp120 CD4-binding pocket. Complexes are shown as ribbon-ball-stick representations with the side chains of gp120 residues contributing to the compounds' binding shown as sticks. (B-C) Details of TFH-I-070-A6 (B) and TFH-I-116-D1 (C) interaction with gp120 Asp³⁶⁸. The closest distances between each compound and the side-chain atoms of Asp³⁶⁸ are shown as dashes. (D) Analysis of the compounds' binding interface. The relative contribution of the indicated gp 120 residue to each compound binding is shown as the buried surface area (BSA), as calculated by PISA. BSA represents the solvent-accessible surface area of the corresponding residue that is buried upon interface formation. (E-G) Compounds ZXC-I-090 and ZXC-I-092 made H-bond with Asp³⁶⁸.

FIG. 4. Anti-CoRBS Ab binding. Anti-CoRBS Ab-17b binding to (A) HIV-1CH58TF, (B) HIV-1JRFL (C) or HIV-1AD8 infected primary CD4+ T cells was done in the presence of 50 μM of the indicated CD4mcs or the same volume of vehicle (DMSO). The median fluorescence intensity (MFI) of 17b binding is reported for the productively-infected cell (p24+) population. Data shown are the mean±SD of at least three independent experiments. Statistical significance was evaluated using an un-paired t test (*, p<0.05; **, p<0.01; ***, p<0.001; ****, p<0.0001; ns, not significant).

FIG. 5. Small CD4-mimetic neutralization of viral particles. Neutralization of VSV-G pseudo-virus (A), HIV-1CH58TF (B), HIV-1JRFL (C) or HIV-1AD8 (D) virus was done with the indicated amounts of different compounds or the same volume of DMSO in TZM-bl cells. luciferase activity (RLU—relative light units) was measured. Relative infectivity was calculated as the percentage of the value seen in the absence of compound. Cell viability for TZM-bl ((E), left) or primary CD4+ T cells ((FE), right) with the different CD4mc was measured by CellTiter-Glo One Solution assay for the quantitation of ATP presented in live cells. Data shown are the mean±SD of at least three independent experiments.

FIG. 6. Piperidine CD4mc analogues sensitize HIV-1-infected cells to ADCC. Primary CD4 T cells were infected with HIV-1CH58TF (A,D), HIV-1JRFL (B,E) or HIV-1AD8 (C,F). HIV+plasma (1:1000 diluted) was used for staining (A-C) or ADCC (D-F) in the presence of 50 μM different compounds or with an equivalent volume of vehicle (DMSO). An Alexa Fluor 647-conjugated anti-human IgG secondary Ab was then used for fluorescent labeling (A-C). Median fluorescence intensity (MFI) in the presence of compounds or that of DMSO was shown; For ADCC, infected cells were used as target cells in a FACS-based ADCC assay that measures the killing of infected (p24+ cells). The assay determines susceptibility to ADCC mediated by a 1/1000 dilution of plasma from HIV-1-infected individuals in the presence of 50 μM different compounds or with an equivalent volume of vehicle (DMSO). (G-I) The correlation between cell surface staining with HIV+ plasma and ADCC was calculated using the Pearson r rank correlation. Data shown are the mean±SD of at least three independent experiments (plasma from more than 5 HIV-1-infected individuals). Statistical significance was evaluated using an unpaired t test (*, p<0.05; **, p<0.01; ***, p<0.001; ****, p<0.0001; ns, not significant).

FIG. 7. BLI competition assay. (A) BLI measurements of tested CD4mcs competition with sCD4 binding to LMHT gp 120_{CRF01_AE} coreₑ enables determination of the concentration required to compete out 50% of CD4-Coreₑ binding (C₅₀). (B) Correlation of ADCC activity against HIV-1_{AD8} and C₅₀ from the sCD4 competition assays. (C) Correlation of neutralization of HIV_{CH58TF} (IC₅₀) and C₅₀ from the sCD4 competition assays.

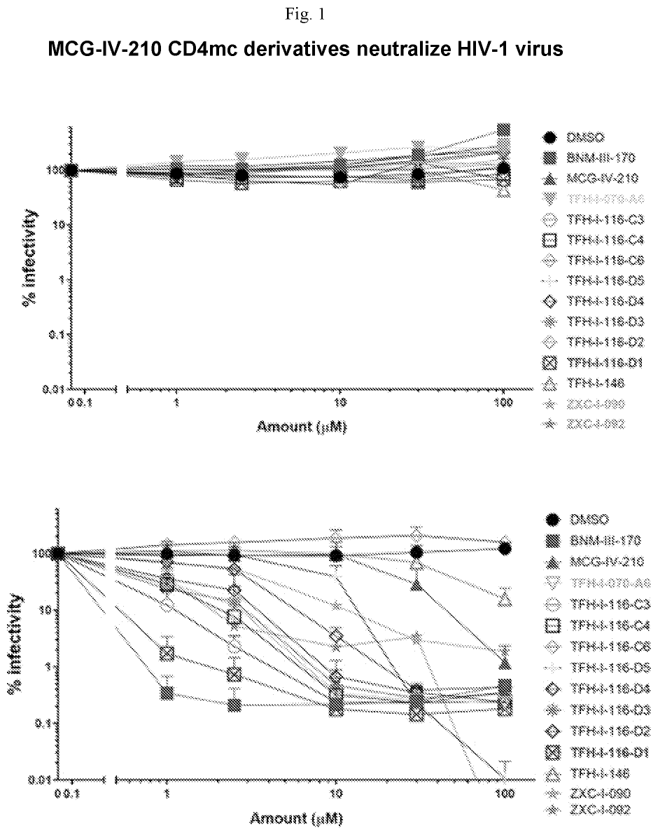

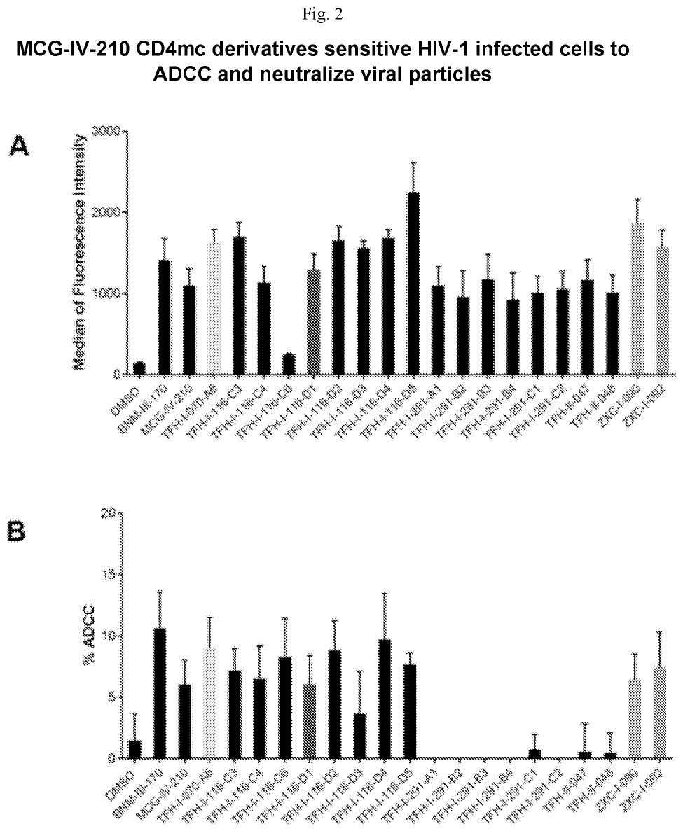

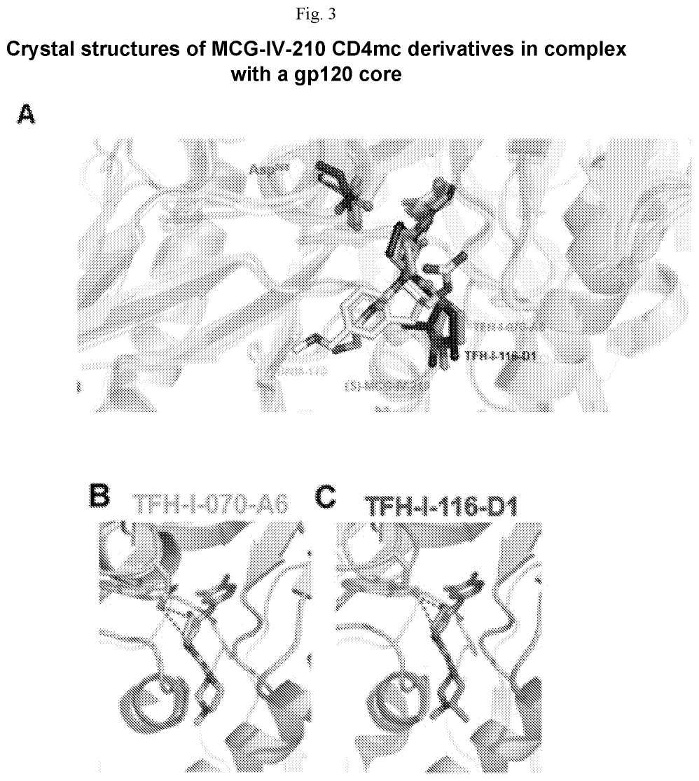

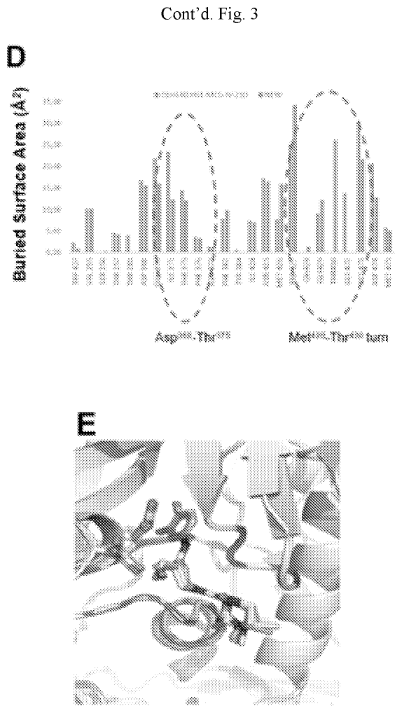

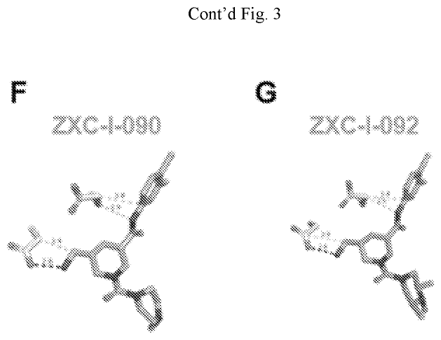

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination. Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

The term “administering” means either directly administering to the subject a compound, or composition comprising that compound, of the present invention. In other aspects, “administering” refers to administering a prodrug, derivative or analog or a compound of the present invention, which will form an equivalent amount of the compound within the body.

As used herein, the term “stereoisomers” refers to compounds which have identical chemical constitution but differ with regard to the arrangement of the atoms or groups in space, e.g., enantiomers, diastereomers, tautomers.

The terms “patient” and “subject” is used interchangeably throughout the specification to describe an animal, for example, a mammal, to whom treatment, including prophylactic treatment, with the compositions according to the present disclosure is provided. Mammals that can be treated using the methods of the disclosure include rodents such as mice, rats, rabbits, guinea pigs, and the like, as well as domesticated animals such as dogs, cats, and farm animals such as a horse, cow, sheep, etc. In other aspects, the mammal is a human.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

As employed above and throughout the disclosure the term “therapeutically effective amount” refers to an amount effective, at dosages, and for periods of time necessary, to achieve the desired result with respect to the treatment of the relevant disorder, condition, or side effect. It will be appreciated that the effective amount of compounds of the present invention will vary from subject to subject.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, trifluoroacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. See for example, Remington, J. P. (2020). Remington, the science and practice of pharmacy, Elsevier Science.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “C₁₋₆alk” refers to an aliphatic linker having 1, 2, 3, 4, 5, or 6carbon atoms and includes, for example, —CH₂—, —CH(CH₃)—, —CH(CH₃)—CH₂—, and —C(CH₃)₂—. The term “-C₀alk-” refers to a bond.

As used herein, the term “alkyl”, by itself or as part of another substituent, means, unless otherwise stated, a straight or branched chain hydrocarbon radical having up to twelve carbon atoms. In some embodiments, the number of carbon atoms is designated (i.e., C₁-C₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is a C₁-C₆ alkyl; in some embodiments, it is a C₁-C₄ alkyl.

The terms “halo” or “halogen”, by itself or as part of another substituent, means a fluorine, chlorine, bromine, or iodine atom.

The term “haloalkyl” refers to an alkyl moiety wherein one or more hydrogens has been replaced with one or more halogen atoms. Examples include CF₃, CH₂CH₃, and the like.

The term “aryl” as used herein refers to a single, all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2, 3 or 4 rings) having about 9 to 12 carbon atoms in which at least one ring is aromatic and wherein the other rings may be aromatic or not aromatic. Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the aromatic ring. Non-limiting examples of aryl groups include, but are not limited to, phenyl, indenyl, naphthyl, 1,2,3,4-tetrahydronaphthyl, and the like

The term “substituted”, as used in combination with a substituent defined herein, means that the substituent is substituted with one or more suitable functional groups or other substituents as provided herein. For example, a substituent may be optionally substituted with one or more of: halo, cyano, C₁₋₆ alkyl, aryl, benzyl, or the like.

The disclosure is directed to compounds of Formula I, stereoisomers of Formula I, and pharmaceutically acceptable salts thereof. . . . While not wishing to be bound by any particular theory, the compounds of formula I are believed to be CD4 mimetic compounds (CD4mc) that open Env, thus allowing enhanced recognition and binding by antibodies resulting in the sensitization of HIV-1 infected cells to ADCC.

This disclosure is directed to compounds of Formula II, as well as stereoisomers thereof, and pharmaceutically acceptable salts thereof:

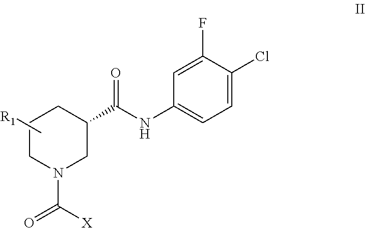

According to the disclosure, R₁ is H, C₁₋₆alkN(R)₂, C₁₋₆alkOR′, or C₁₋₆alk(O)N(R)₂, wherein each R is independently H or C₁₋₆alkyl; R′ is independent H or C₁₋₆alkyl. In some aspects, R₁ is H. In other aspects, R₁ is C₁₋₆alkN(R)₂. In some aspects, R₁ is C₁₋₃alkN(R)₂. In some aspects, R₁ is C₁alkN(R)₂. In some aspects, R₁ is C₂alkN(R)₂. In some aspects, R₁ is C₃alkN(R)₂. In some aspects, each R is independently H or C₁₋₃alkyl. In some aspects, each R is H. In some aspects, one R is H and the other is C₁₋₃alkyl. In some aspects, R₁ is C₁alkNH₂ (—CH₂NH₂). In other aspects, R₁ is C₁₋₆alkOR′, wherein R′ is independent H or C₁₋₆alkyl. In some aspects, R₁ is C₁₋₃alkOR′. In some aspects, R₁ is C₁alkOR′. In some aspects, R₁ is C₂alkOR′. In some aspects, R₁ is C₃alkOR′. In some aspects, R₁ is CH₂OH. In some aspects, R₁ is CH₂CH₂OH. In some aspects, R₁ is CH₂CH₂CH₂OH. In some aspects, C₁₋₆alk(O)N(R)₂, wherein each R is independently H or C₁₋₆alkyl. In some aspects, R₁ is C₁alk(O)N(R)₂. In some aspects, R₁ is C₂alk(O)N(R)₂. In some aspects, R₁ is C₃alk(O)N(R)₂. In some aspects, each R is independently H or C₁₋₃alkyl. In some aspects, each R is H. In some aspects, one R is H and the other is C₁₋₃alkyl. In some aspects, R₁ is C(O)—NH₂.

According to the disclosure, X is

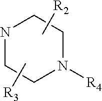

or a bicyclic ring system; wherein the bicyclic ring system is a 9 membered bicyclic aromatic ring system consisting of a 6 membered ring fused with a 5-membered ring; wherein the 5-membered ring comprises one, two or three heteroatoms each independently selected from O, S, and N; the bicyclic aromatic ring system is attached to the remainder of the molecule via a ring carbon atom of the 5- or 6-membered ring or a ring nitrogen atom of the 5-membered ring; the bicyclic ring system is optionally substituted on the carbon atoms with one, two, three, or four substituents each independently halo, oxo, —OH, —NH2, —NH-C₁₋₄alkyl, C₂₋₆alkenyl, or C₁₋₄alkyl. In some aspects, X is

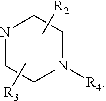

In some aspects, X is the bicyclic ring system. In some aspects, X is

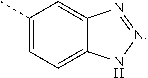

According to the disclosure, R₂ is H, halo, C₀₋₆alk-aryl, substituted C₀₋₆alk-aryl, C₁₋₆alkyl, substituted C₁₋₆alkyl, or C₁₋₆haloalkyl. In some aspects, R₂ is H.

In some aspects, R₂ is halo, for example, F, Cl, or Br. In some aspects, R₂ is F. In some aspects, R₂ is Cl. In some aspects, R₂ is Br.

In some aspects, R₂ is C₀₋₆alk-aryl. In some aspects, R₂ is C₀₋₃alk-aryl. In some aspects R₂ is C₀alk-aryl. In some aspects, R₂ is C₁alk-aryl. In some aspects R₂ is C₂alk-aryl. In some aspects, R₂ is C₀₋₆alk-phenyl.

In some aspects, R₂ is substituted C₀₋₆alk-aryl. In these aspects, the aryl moiety may be substituted. In other aspects, when present, the C₁₋₆alk moiety is substituted. In other aspects, the aryl and the C₁₋₆alk moiety are each independently substituted. In some aspects, R₂ is substituted C₀₋₃alk-aryl. In some aspects R₂ is substituted C₀alk-aryl. In some aspects, R₂ is substituted C₁alk-aryl. In some aspects R₂ is substituted C₂alk-aryl. In some aspects, R₂ is substituted C₀₋₆alk-phenyl

In some aspects, R₂ is C₁₋₆alkyl. In some aspects, R₂ is C₁₋₃alkyl. In some aspects R₂ is C₁alkyl. In some aspects, R₂ is C₂alkyl. In some aspects R₂ is C₃alkyl. In some aspects, R₂ is —CH₃.

In some aspects, R₂ is substituted C₁₋₆alkyl. In some aspects, R₂ is substituted C₁₋₃alkyl. In some aspects R₂ is substituted C₁alkyl. In some aspects, R₂ is substituted C₂alkyl. In some aspects R₂ is substituted C₃alkyl.

In some aspects, R₂ is C₁₋₆haloalkyl. In some aspects, R₂ is C₁₋₃haloalkyl. In some aspects R₂ is C₁haloalkyl. In some aspects, R₂ is C₂haloalkyl. In some aspects R₂ is C₃haloalkyl. In some aspects, R₂ is —CF₃.

According to the disclosure, R₃ is H, halo, C₀₋₆alk-aryl, substituted C₀₋₆alk-aryl, C₁₋₆alkyl, substituted C₁₋₆alkyl, or C₁₋₆haloalkyl. In some aspects, R₃ is H.

In some aspects, R₃ is halo, for example, F, Cl, or Br. In some aspects, R₃ is F. In some aspects, R₃ is Cl. In some aspects, R₃ is Br.

In some aspects, R₃ is C₀₋₆alk-aryl. In some aspects, R₃ is C₀₋₃alk-aryl. In some aspects R₃ is C₀alk-aryl. In some aspects, R₃ is C₁alk-aryl. In some aspects, R₃ is C₂alk-aryl. In some aspects, R₃ is C₀₋₆alk-phenyl.

In some aspects, R₃ is substituted C₀₋₆alk-aryl. In these aspects, the aryl moiety may be substituted. In other aspects, when present, the C₁₋₆alk moiety is substituted. In other aspects, the aryl and the C₁₋₆alk moiety are each independently substituted. In some aspects, R₃ is substituted C₀₋₃alk-aryl. In some aspects R₃ is substituted C₀alk-aryl. In some aspects, R₃ is substituted C₁alk-aryl. In some aspects R₃ is substituted C₂alk-aryl. In some aspects, R₃ is substituted C₀₋₆alk-phenyl.

In some aspects, R₃ is C₁₋₆alkyl. In some aspects, R₃ is C₁₋₃alkyl. In some aspects, R₃ is C₁alkyl. In some aspects, R₃ is C₂alkyl. In some aspects, R₃ is C₃alkyl. In some aspects, R₃ is —CH₃.

In some aspects, R₃ is substituted C₁₋₆alkyl. In some aspects R₃ is substituted

C₁₋₃alkyl. In some aspects, R₃ is substituted C₁alkyl. In some aspects, R₃ is substituted C₂alkyl. In some aspects R₃ is substituted C₃alkyl.

In some aspects, R₃ is C₁₋₆haloalkyl. In some aspects, R₃ is C₁₋₃haloalkyl. In some aspects R₃ is C₁haloalkyl. In some aspects, R₃ is C₂haloalkyl. In some aspects R₃ is C₃haloalkyl. In some aspects, R₃ is —CF₃.

According to the disclosure, R₄ is H or C₁₋₆alkyl. In some aspects, R₄ is H. In some aspects, R₄ is C₁₋₆alkyl. In some aspects, R₄ is —CH₃, —CH₂CH₃, and the like. In other aspects, R₄ is C₁₋₆alkylCOOH, or C(O)C₁₋₆alkN(R″)₂, wherein R″ is independent H or C₁₋₆alkyl.

In some aspects, R₄ is C₁₋₃alkCOOH. In some aspects, R₄ is C₁alkCOOH. In some aspects, R₄ is C₂alkCOOH. In some aspects, R₄ is C₃alkCOOH.

In some aspects, R₄ is C(O) C₁₋₃alkN(R″)₂. In some aspects, R₄ is C(O) C₁alkN(R″)₂. In some aspects, R₄ is C(O) C₂alkN(R″)₂. In some aspects, R₄ is C(O) C₃alkN(R″)₂. In some aspects, R₄ is —C(O)CH₂NH₂.

The disclosure is directed to compounds of formula I, as well as stereoisomers thereof, and pharmaceutically acceptable salts thereof:

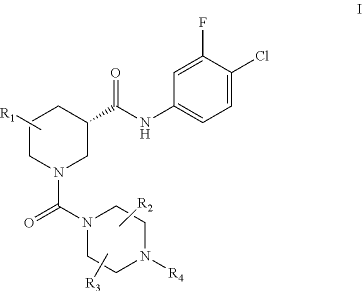

According to the disclosure, R₁ is H, or C₁₋₆alkN(R)₂, wherein each R is independently H or C₁₋₆alkyl; R₂ is H, halo, C₀₋₆alk-aryl, substituted C₀₋₆alk-aryl, C₁₋₆alkyl, substituted C₁₋₆alkyl, or C₁₋₆haloalkyl; R₃ is H, halo, C₀₋₆alk-aryl, substituted C₀₋₆alk-aryl, C₁₋₆alkyl, substituted C₁₋₆alkyl, or C₁₋₆haloalkyl; and R₄ is H or C₁₋₆alkyl.

According to the disclosure, R₁ is H or C₁₋₆alkN(R)₂, wherein each R is independently H or C₁₋₆alkyl. In some aspects, R₁ is H. In other aspects, R₁ is C₁₋₆alkN(R)₂. In some aspects, R₁ is C₁₋₃alkN(R)₂. In some aspects, R₁ is C₁alkN(R)₂. In some aspects, R₁ is C₂alkN(R)₂. In some aspects, R₁ is C₃alkN(R)₂. In some aspects, each R is independently H or C₁₋₃alkyl. In some aspects, each R is H. In some aspects, one R is H and the other is C₁₋₃alkyl. In some aspects, R₁ is C₁alkNH₂ (—CH₂NH₂). In other aspects, R₁ is C₁₋₆alkOR′, wherein R′ is independent H or C₁₋₆alkyl. In some aspects, R₁ is C₁₋₃alkOR′. In some aspects, R₁ is C₁alkOR′. In some aspects, R₁ is C₂alkOR′. In some aspects, R₁ is C₃alkOR′. In some aspects, R₁ is CH₂OH. In some aspects, R₁ is CH₂CH₂OH. In some aspects, R₁ is CH₂CH₂CH₂OH. In some aspects, C₁₋₆alk(O)N(R)₂, wherein each R is independently H or C₁₋₆alkyl. In some aspects, R₁ is C₁alk(O)N(R)₂. In some aspects, R₁ is C₂alk(O)N(R)₂. In some aspects, R₁ is C₃alk(O)N(R)₂. In some aspects, each R is independently H or C₁₋₃alkyl. In some aspects, each R is H. In some aspects, one R is H and the other is C₁₋₃alkyl. In some aspects, R₁ is C(O)—NH₂.

According to the disclosure, R₂ is H, halo, C₀₋₆alk-aryl, substituted C₀₋₆alk-aryl, C₁₋₆alkyl, substituted C₁₋₆alkyl, or C₁₋₆haloalkyl. In some aspects, R₂ is H.

In some aspects, R₂ is halo, for example, F, Cl, or Br. In some aspects, R₂ is F. In some aspects, R₂ is Cl. In some aspects, R₂ is Br.

In some aspects, R₂ is C₀₋₆alk-aryl. In some aspects, R₂ is C₀₋₆alk-aryl. In some aspects R₂ is C₀alk-aryl. In some aspects, R₂ is C₁alk-aryl. In some aspects R₂ is C₂alk-aryl. In some aspects, R₂ is C₀₋₆alk-phenyl.

In some aspects, R₂ is substituted C₀₋₆alk-aryl. In these aspects, the aryl moiety may be substituted. In other aspects, when present, the C₁₋₆alk moiety is substituted. In other aspects, the aryl and the C₁₋₆alk moiety are each independently substituted. In some aspects, R₂ is substituted C₀₋₃alk-aryl. In some aspects R₂ is substituted C₀alk-aryl. In some aspects, R₂ is substituted C₁alk-aryl. In some aspects R₂ is substituted C₂alk-aryl. In some aspects, R₂ is substituted C₀₋₆alk-phenyl

In some aspects, R₂ is C₁₋₆alkyl. In some aspects, R₂ is C₁₋₃alkyl. In some aspects R₂ is C₁alkyl. In some aspects, R₂ is C₂alkyl. In some aspects R₂ is C₃alkyl. In some aspects, R₂ is —CH₃.

In some aspects, R₂ is substituted C₁₋₆alkyl. In some aspects, R₂ is substituted C₁₋₃alkyl. In some aspects R₂ is substituted C₁alkyl. In some aspects, R₂ is substituted C₂alkyl. In some aspects R₂ is substituted C₂alkyl.

In some aspects, R₂ is C₁₋₆haloalkyl. In some aspects, R₂ is C₁₋₃haloalkyl. In some aspects R₂ is C₁haloalkyl. In some aspects, R₂ is C₂haloalkyl. In some aspects R₂ is C₃haloalkyl. In some aspects, R₂ is —CF₃.

According to the disclosure, R₃ is H, halo, C₀₋₆alk-aryl, substituted C₀₋₆alk-aryl, C₁₋₆alkyl, substituted C₁₋₆alkyl, or C₁₋₆haloalkyl. In some aspects, R₃ is H.

In some aspects, R₃ is halo, for example, F, Cl, or Br. In some aspects, R₃ is F. In some aspects, R₃ is Cl. In some aspects, R₃ is Br.

In some aspects, R₃ is C₀₋₆alk-aryl. In some aspects, R₃ is C₀₋₃alk-aryl. In some aspects R₃ is C₀alk-aryl. In some aspects, R₃ is C₁alk-aryl. In some aspects, R₃ is C₂alk-aryl. In some aspects, R₃ is C₀₋₆alk-phenyl.

In some aspects, R₃ is substituted C₀₋₆alk-aryl. In these aspects, the aryl moiety may be substituted. In other aspects, when present, the C₁₋₆alk moiety is substituted. In other aspects, the aryl and the C₁₋₆alk moiety are each independently substituted. In some aspects, R₃ is substituted C₀₋₃alk-aryl. In some aspects R₃ is substituted C₀alk-aryl. In some aspects, R₃ is substituted C₁alk-aryl. In some aspects R₃ is substituted C₂alk-aryl. In some aspects, R₃ is substituted C₀₋₆alk-phenyl.

In some aspects, R₃ is C₁₋₆alkyl. In some aspects, R₃ is C₁₋₃alkyl. In some aspects, R₃ is C₁alkyl. In some aspects, R₃ is C₂alkyl. In some aspects, R₃ is C₃alkyl. In some aspects, R₃ is —CH₃.

In some aspects, R₃ is substituted C₁₋₆alkyl. In some aspects R₃ is substituted C₁₋₃alkyl. In some aspects, R₃ is substituted C₁alkyl. In some aspects, R₃ is substituted C₂alkyl. In some aspects R₃ is substituted C₃alkyl.

In some aspects, R₃ is C₁₋₆haloalkyl. In some aspects, R₃ is C₁₋₃haloalkyl. In some aspects R₃ is C₁haloalkyl. In some aspects, R₃ is C₂haloalkyl. In some aspects R₃ is C₃haloalkyl. In some aspects, R₃ is —CF₃.

According to the disclosure, R₄ is H or C₁₋₆alkyl. In some aspects, R₄ is H. In some aspects, R₄ is C₁₋₆alkyl. In some aspects, R₄ is —CH₃, —CH₂CH₃, and the like. In other aspects, R₄ is C₁₋₆alkylCOOH, or C(O) C₁₋₆alkN(R″)₂, wherein R″ is independent H or C₁₋₆alkyl.

In some aspects, R₄ is C₁₋₃alkCOOH. In some aspects, R₄ is C₁alkCOOH. In some aspects, R₄ is C₂alkCOOH. In some aspects, R₄ is C₃alkCOOH.

In some aspects, R₄ is C(O) C₁₋₃alkN(R″)₂. In some aspects, R₄ is C(O) C₁alkN(R″)₂. In some aspects, R₄ is C(O)C₂alkN(R″)₂. In some aspects, R₄ is C(O) C₃alkN(R″)₂. In some aspects, R₄ is —C(O)CH₂NH₂.

In certain aspects, the compound is a compound of Formula IA, stereoisomers of Formula IA, and pharmaceutically acceptable salts thereof.

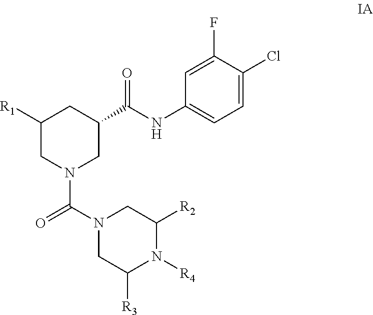

In some embodiments the compound of formula I is TFH-I-070-A6, a stereoisomer of compound TFH-I-070-A6, or a pharmaceutically acceptable salt thereof.

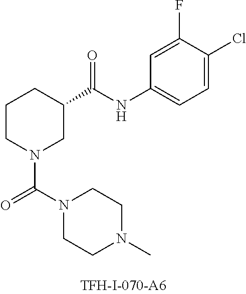 [SMILES: CN1CCN(C(=O)N2CCC[C@H](C(=O)Nc3ccc(Cl)c(F)c3)C2)CC1]

In some embodiments the compound of formula I is TFH-I-070-A6, a stereoisomer of compound TFH-I-070-A6, or a pharmaceutically acceptable salt thereof.

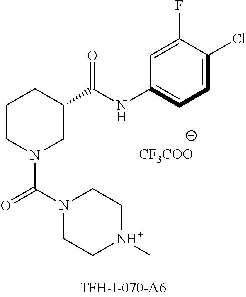

In some embodiments the compound of formula I is TFH-I-116-D1, a stereoisomer of compound TFH-I-116-D1, or a pharmaceutically acceptable salt thereof.

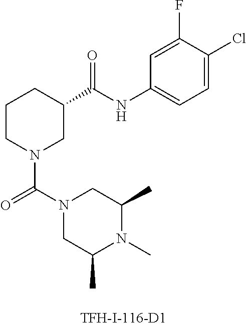 [SMILES: C[C@@H]1CN(C(=O)N2CCC[C@H](C(=O)Nc3ccc(Cl)c(F)c3)C2)C[C@H](C)N1C]

In some embodiments the compound of formula I is TFH-I-116-D1, a stereoisomer of compound TFH-I-116-D1, or a pharmaceutically acceptable salt thereof.

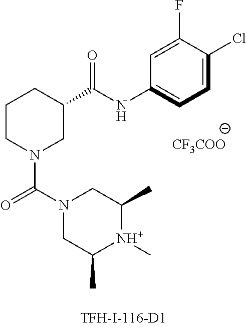

In some embodiments the compound of formula I is ZXC-I-090, a stereoisomer of compound ZXC-I-090, or a pharmaceutically acceptable salt thereof.

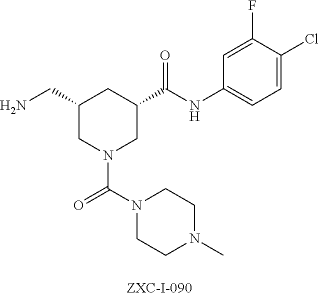 [SMILES: CN1CCN(C(=O)N2C[C@H](CN)C[C@H](C(=O)Nc3ccc(Cl)c(F)c3)C2)CC1]

In some embodiments the compound of formula I is ZXC-I-090, a stereoisomer of compound ZXC-I-090, or a pharmaceutically acceptable salt thereof.

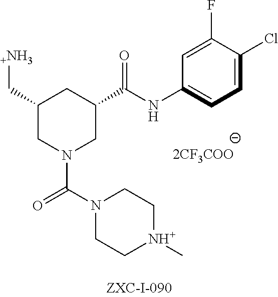

In some embodiments the of formula I is ZXC-I-092, a stereoisomer of compound ZXC-I-092, or a pharmaceutically acceptable salt thereof.

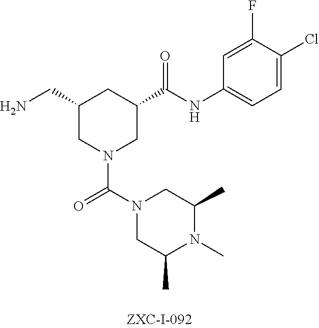 [SMILES: C[C@@H]1CN(C(=O)N2C[C@H](CN)C[C@H](C(=O)Nc3ccc(Cl)c(F)c3)C2)C[C@H](C)N1C]

In some embodiments the of formula I is ZXC-I-092, a stereoisomer of compound ZXC-I-092, or a pharmaceutically acceptable salt thereof.

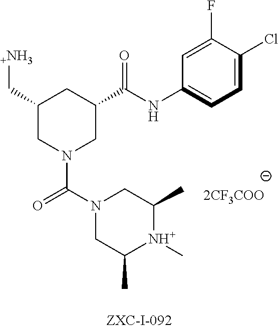

In some embodiments the of formula I is TFH-II-128, a stereoisomer of compound TFH-II-128, or a pharmaceutically acceptable salt thereof.

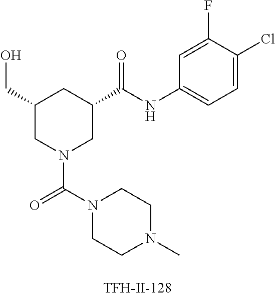 [SMILES: CN1CCN(C(=O)N2C[C@H](CO)C[C@H](C(=O)Nc3ccc(Cl)c(F)c3)C2)CC1]

In some embodiments the of formula I is TFH-I-151, a stereoisomer of compound TFH-I-151, or a pharmaceutically acceptable salt thereof.

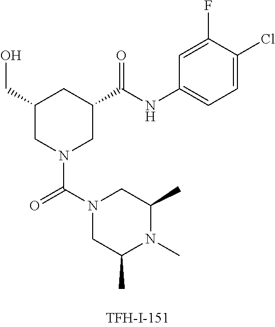 [SMILES: C[C@@H]1CN(C(=O)N2C[C@H](CO)C[C@H](C(=O)Nc3ccc(Cl)c(F)c3)C2)C[C@H](C)N1C]

In some embodiments the of formula I is ZXC-I-258, a stereoisomer of compound ZXC-I-258, or a pharmaceutically acceptable salt thereof.

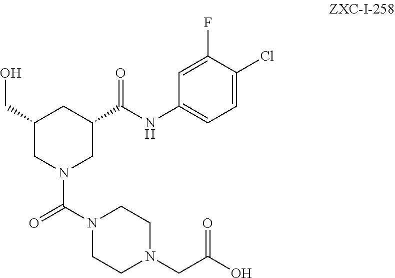 [SMILES: O=C(O)CN1CCN(C(=O)N2C[C@H](CO)C[C@H](C(=O)Nc3ccc(Cl)c(F)c3)C2)CC1]

In some embodiments, the of formula I is ZXC-I-258 (DL-I-102), a stereoisomer of compound ZXC-I-258 (DL-I-102), or a pharmaceutically acceptable salt thereof.

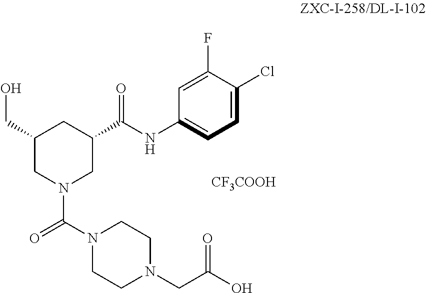

In some embodiments the of formula I is ZXC-I-257, a stereoisomer of compound ZXC-I-257, or a pharmaceutically acceptable salt thereof.

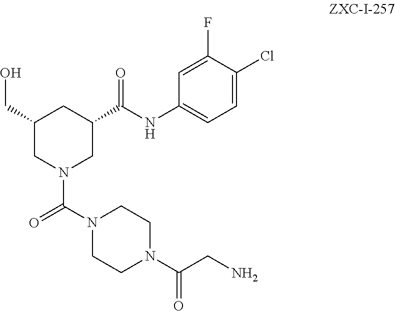 [SMILES: NCC(=O)N1CCN(C(=O)N2C[C@H](CO)C[C@H](C(=O)Nc3ccc(Cl)c(F)c3)C2)CC1]

In some embodiments, the of formula I is ZXC-I-257/DL-I-101, a stereoisomer of compound ZXC-I-257/DL-I-101, or a pharmaceutically acceptable salt thereof.

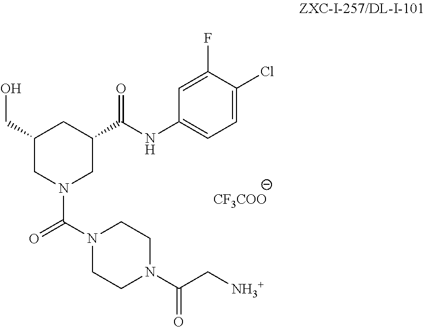

In some embodiments, the of formula I is DL-I-211, a stereoisomer of compound DL-I-211, or a pharmaceutically acceptable salt thereof.

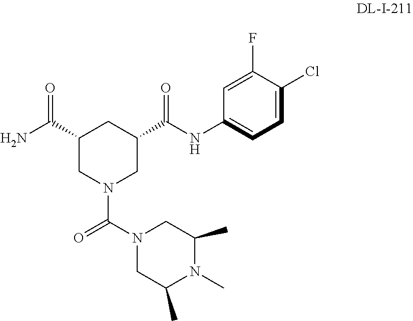

In some embodiments, the of formula I is DL-I-211, a stereoisomer of compound DL-I-211, or a pharmaceutically acceptable salt thereof.

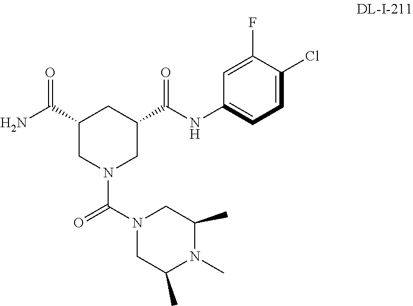

In some embodiments, the of formula II is HZ-IV-16, a stereoisomer of compound HZ-IV-16, or a pharmaceutically acceptable salt thereof.

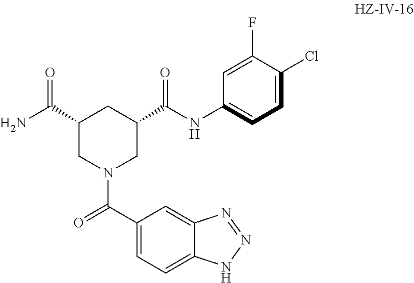

In some aspects, the compounds of the disclosure are useful in treating or preventing HIV-1 replication in a mammal. In these aspects, a compound of the disclosure is administered to the mammal in need of HIV-1 replication treatment or prevention.

In some aspects, the compounds of the disclosure are useful in preventing transmission of HIV-1 to a mammal. In these aspects, a compound of the disclosure is administered to the mammal in need.

In some aspects, compounds useful in the methods of the disclosure include those described in, for example, Grenier, M. C. et al. “Optimization of small molecules that sensitize HIV-1 infected cells to antibody-dependent cellular cytotoxicity” ACS Med. Chem. Lett. 2020, 11, 371-378, the disclosures of which are incorporated by reference herein.

In some aspects, the compounds of the disclosure are used in combination with one or more antiretrovirals that are known in the art.

Compounds of the disclosure can be combined with pharmaceutically acceptable excipients to produce pharmaceutical compositions. Pharmaceutical compositions are typically formulated to provide a therapeutically effective amount of a compound of the present disclosure as the active ingredient, or a stereoisomer thereof, or a pharmaceutically acceptable salt, thereof in combination with one or more pharmaceutically acceptable excipients, carriers, including inert solid diluents and fillers, diluents, including sterile aqueous solution and various organic solvents, permeation enhancers, solubilizers and adjuvants.

The compounds of the disclosure can be prepared using synthetic techniques known in the art. Exemplary methods of preparing compounds of the disclosure are summarized in schemes A and B.

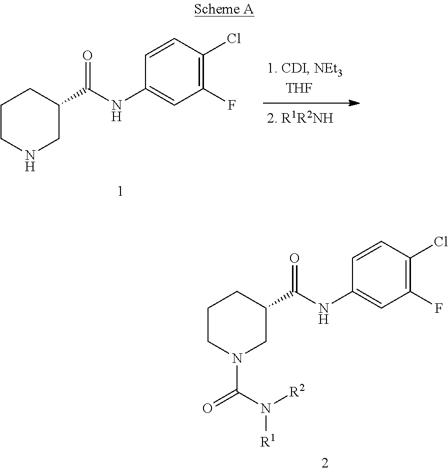

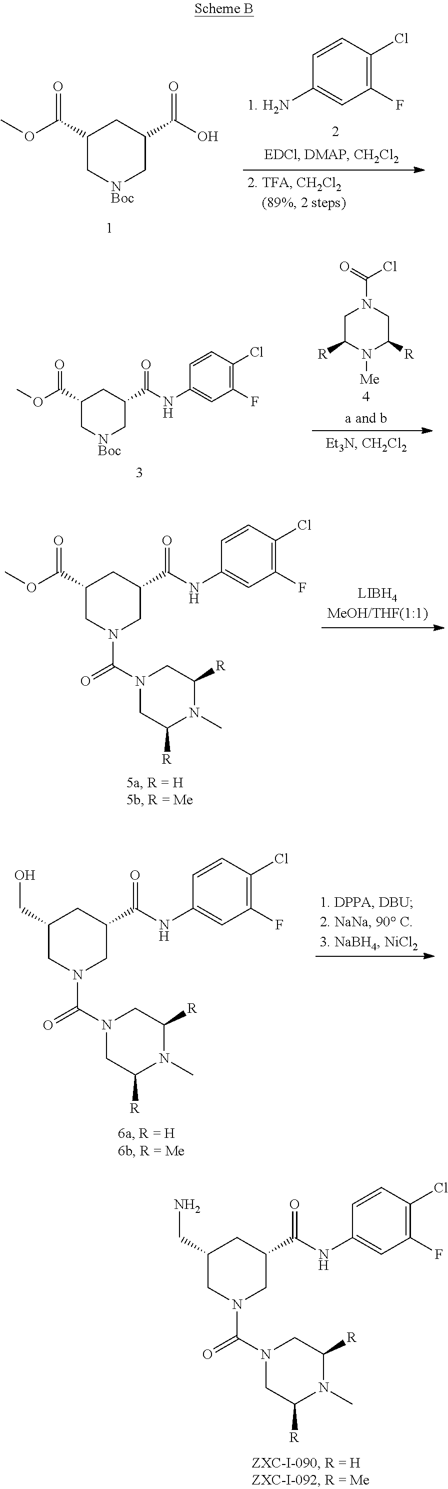

### EXAMPLES

The following examples are exemplary, only and should not be construed as limiting the scope of the invention.

### Example 1

General Procedure to synthesize TFH analogs

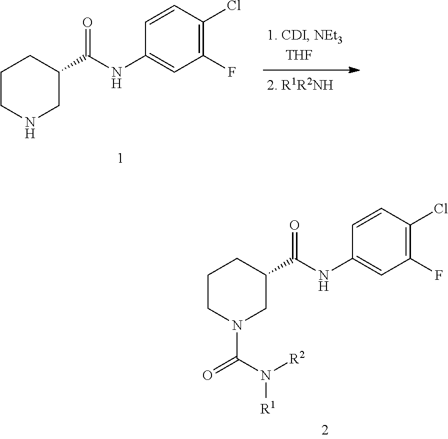

Parallel Synthesis I: To a precooled solution of CDI (395 mg, 2.44 mmol, 1.2 eq.) in THF (0.5 M) at 0° C. was added piperidine 1 (520 mg, 2.03 mmol, 1.0 eq) potion-wise. The reaction mixture was stirred for 1 hr at 0° C. The reaction mixture was then brought into a glove box and evenly distributed into 24 vials. To the 24 vials were added separately to the corresponding amines (2.0 eq.). The vials are sealed and heated to 80° C. for 16 hours. After cooled down to rt., the 24 samples were analyzed by LCMS to confirm the formation of the desired product. Concentration and high-performance liquid chromatography purification afforded the corresponding product.

(S)-MCG-IV-210 and (+)-BNM-III-170 are shown below.

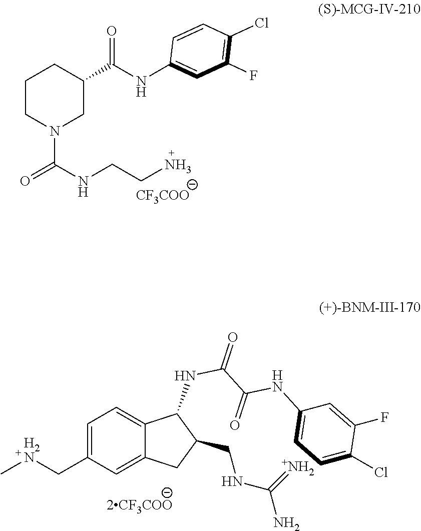

### Example 2

### TFH-I-070-A6 Characterization Data

(S)-4-(3-((4-chloro-3-fluorophenyl)carbamoyl)piperidine-1-carbonyl)-1-methylpiperazin-1-ium, 2,2,2-trifluoro-acetate ¹H NMR (600 MHz, MeOD) δ 7.73 (dd, J=11.6, 2.4 Hz, 1H), 7.40 (t, J=8.5 Hz, 1H), 7.28-7.26 (m, 1H), 3.84-3.80 (m, 3H), 3.73-3.63 (m, 1H), 3.58-3.46 (m, 2H), 3.24-3.13 (m, 5H), 3.09-3.04 (m, 1H), 2.94 (s, 3H), 2.61 (dddd, J=10.5, 10.5, 3.8, 3.8 Hz, 1H), 2.09-2.05 (m, 1H), 1.86-1.79 (m, 2H), 1.62-1.55 (m, 1H). ¹³C NMR (126 MHz, MeOD) δ 174.47, 164.47, 159.13 (d, J=245.2 Hz), 140.39 (d, J=10.0 Hz), 117.29 (d, J=3.5 Hz), 116.00 (d, J=18.1 Hz), 109.20 (d, J=26.1 Hz), 131.56, 54.13, 45.42, 44.54, 43.72, 40.41, 29.04, 25.44. LRMS(ESI) m/z 384.1490 [calcd for C₁₈H₂₅ClFN₄O₂ (M+H)⁺] 383.1645.

### Example 3

### General Procedure to Synthesize ZXC Analogs

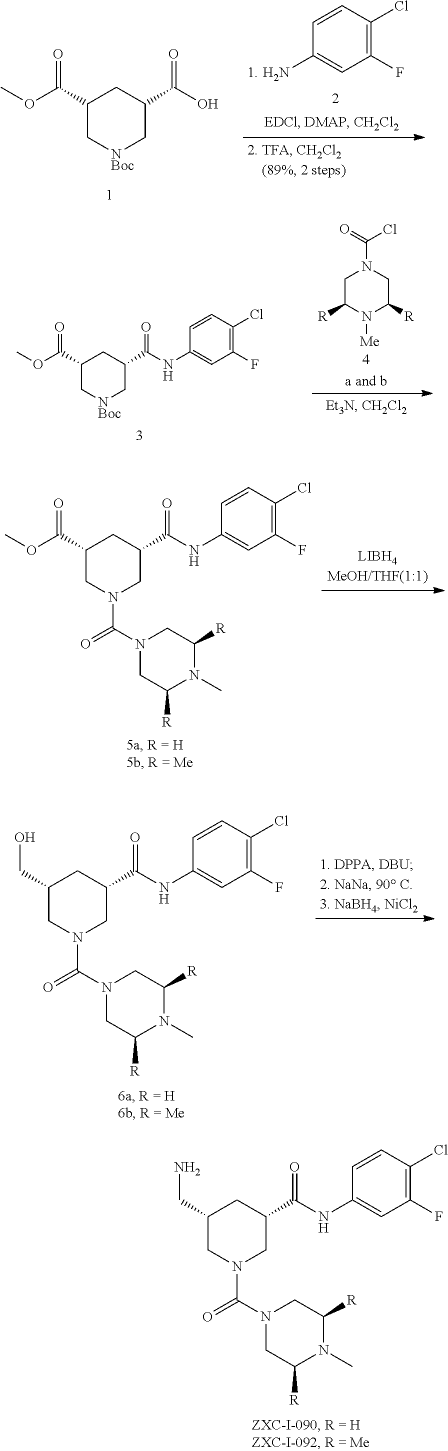

An EDC coupling reaction of compound 1 with 4-chloro-3-fluoroaniline 2 gives 3. Subsequently, removal of the Boc group and installation of the piperazine urea with carbamyl chloride (4a and 4b) resulted in the formation of (5a and 5b). At this stage, LiBH₄ reduction 5 (a and b) in the co-solvent of H₂O and MeOH successfully gave alcohol 6 (a and b). With 6 in hand, a sequence of phosphate formation by treatment of DPPA, azide replacement of phosphate, and the subsequent azide reduction by a mixture of NiCl₂ and NaBH₄ lead to the formation of the desired ZXC-amino analog ZXC-I-090 and ZXC-I-092 respectively.

The compounds of the disclosure sensitize primary CD4+ T cells infected with a clinically relevant transmitted/founder virus (CH58TF) to HIV+ serum-mediated ADCC, as established by cytometry-based assays. Richard, J.; Veillette, M.; Brassard, N.; Iyer, S. S.; Roger, M.; Martin, L.; Pazgier, M.; Scho\ddot{ }n, A.; Freire, E.; Routy, J.-P.; et al. CD4Mimetics Sensitize HIV-1-Infected Cells to ADCC. Proc. Natl. Acad. Sci. U. S. A. 2015, 112 (20), e2687-94, incorporated by reference herein. See, e.g., Table 1.

TABLE 1
IC50 and % ADCC (Approximate Values):

| | IC50 | | IC50 | % ADCC | % ADCC |
|---|---|---|---|---|---|
| Compound | (CH58) | | (JR-FL) | (CH58) | (JR-FL) |
| TFH-I-070-A6 | 30 | uM | 80 uM | 5% | 3% |
| TFH-I-116-D1 | <1 | uM | 20 uM | 5% | 3% |
| ZXC-I-090 | ~50 | uM | 18 uM | ~3% | ~2% |
| ZXC-I-092 | ~50 | uM | 28 uM | ~3% | ~2% |

### Example 4

### General Procedure to Synthesize TFH and ZXC Analogs:

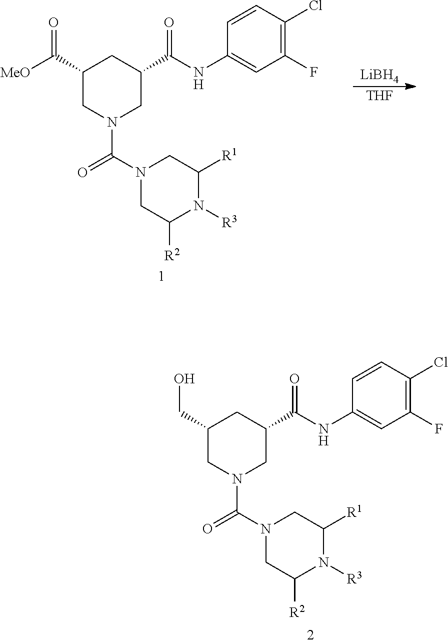

To a precooled solution of 1 (0.4 mmol, 1.0 eq.) in THF (0.5 M) at 0° C. was added lithium borohydride (2.00 mmol, 5.0 eq) portion-wise. The reaction mixture was stirred for 2 hours at 0° C. The crude mixture was analyzed by LCMS to confirm the formation of the desired product. The reaction was quenched by the addition of saturated NH₄Cl (2 mL) and the resulting mixture was extracted with additional DCM (dichloromethane, 2×5 mL). The organic layers were combined, washed with brine (10 mL), dried (MgSO₄), and concentrated. The resulting residue was purified by high-performance liquid chromatography purification to afford corresponding products.

### Example 5

### TFH-I-128 Characterization Data

(3S,5R)-N-(4-chloro-3-fluorophenyl)-5-(hydroxymethyl)-1-(4-methylpiperazine-1-carbonyl)piperidine-3-carboxamide ¹H NMR (600 MHz, MeOD) δ 7.72 (dd, J=11.4, 2.2 Hz, 1H), 7.40 (t, J=8.5 Hz, 1H), 7.30-7.24 (m, 1H), 3.96-3.93 (m, 1H), 3.87 (d, J=12.0 Hz, 1H), 3.81 (t, J=13.9 Hz, 2H), 3.53 (dd, J=11.0, 5.1 Hz, 2H), 3.43 (dd, J=11.0, 7.5 Hz, 2H), 3.45-3.35 (m, 2H), 3.09 (mc, 2H), 3.04 (dd, J=14.6, 11.6 Hz, 1H), 2.98 (d, J=12.7 Hz, 4H), 2.65 (t, J=12.4 Hz, 2H), 2.48 (dddd, J=12.4, 11.9, 3.8, 3.7 Hz, 2H), 2.06 (d, J=12.9 Hz, 1H), 1.79-1.74 (m, 1H), 1.48 (q, J=12.5 Hz, 1H) ¹³C NMR (151 MHz, MeOD) & 172.91, 163.68, 158.54, 130.14, 115.91, 107.81 (d, J=26.0 Hz), 64.00, 58.23, 50.66, 50.13, 43.26, 41.92, 41.78, 38.04, 30.66. LRMS (ESI) m/z calculated for C₁₉H₂₇ClFN₄O₃⁺ [M+H⁺] requires 413.1750; found 413.1744.

### Example 6

### TFH-I-151 Characterization Data:

(3S,5R)-N-(4-chloro-3-fluorophenyl)-5-(hydroxymethyl)-1- ((3S,5R)-3,4,5-trimethylpiperazine-1-carbonyl)piperidine-3-carboxamide ¹H NMR (600 MHz, MeOD) δ 7.73 (dd, J=11.6, 2.4 Hz, 1H), 7.40 (t, J=8.5 Hz, 1H), 7.30-7.24 (m, 1H), 3.94 (d, J=12.8 Hz, 1H), 3.87 (d, J=12.0 Hz, 1H), 3.81 (t, J=13.9 Hz, 2H), 3.53 (dd, J=11.0, 5.1 Hz, 1H), 3.43 (dd, J=11.0, 7.5 Hz, 2H), 3.45-3.35 (m, 1H), 3.09 (m, 1H), 3.04 (dd, J=14.6, 11.6 Hz, 1H), 2.98 (d, J=12.7 Hz, 4H), 2.69 (t, J=12.4 Hz, 1H), 2.65 (dddd, J=12.4, 11.9, 3.8, 3.7 Hz, 1H), 2.06 (d, J=12.9 Hz, 1H), 1.78 (ddt, J=11.4, 7.2, 4.1 Hz, 1H), 1.49 (q, J=12.5 Hz, 1H), 1.45-1.34 (m, 6H). ¹³C NMR (151 MHz, MeOD) δ 172.82, 162.55, 158.54, 156.91, 138.98, 138.91, 130.16, 115.92, 115.89, 114.70, 114.58, 107.90, 107.73, 63.92, 60.12, 60.07, 50.69, 50.49, 49.86, 43.13, 38.05, 36.03, 30.70, 13.47, 13.45. LRMS (ESI) m/z calculated for C₂₁H₃₁ClFN₄O₃⁺ [M+H⁺] requires 441.2063; found 441.2103.

### Example 7

### ZXC-I-257 Characterization Data

(3S,5R)-N-(4-chloro-3-fluorophenyl)-1-(4-glycylpiperazine-1-carbonyl)-5-(hydroxymethyl)piperidine-3-carboxamide ¹H NMR (500 MHz, MeOD) δ 7.72 (dd, J=11.6, 2.4 Hz, 1H), 7.40 (t, J=8.5 Hz, 1H), 7.27 (ddd, J=8.8, 2.3, 1.1 Hz, 1H), 3.96-3.80 (m, 2H), 3.68 (t, J=5.4 Hz, 2H), 3.57-3.39 (m, 4H), 3.39-3.33 (m, 6H), 2.95 (dd, J=13.1, 11.5 Hz, 1H), 2.66 (td, J=12.1, 2.2 Hz, 2H), 2.12-2.02 (m, 1H), 1.86-1.70 (m, 1H), 1.58-1.37 (m, 1H) ¹³C NMR (126 MHz, MeOD) δ 173.05, 164.57, 163.99, 158.84, 156.89, 139.06, 130.29, 116.05, 114.70, 108.05, 107.84, 64.13, 50.22, 46.56, 46.39, 44.07, 43.38, 41.61, 39.69, 38.19, 30.85. LRMS (ESI) m/z calculated for C₂₀H₂₈ClFN₅O₄⁺ [M+H⁺] requires 456.1808; found 456.1816.

### Example 8

### ZXC-I-258 Characterization Data:

2-(4-((3S,5R)-3-((4-chloro-3-fluorophenyl)carbamoyl)-5-(hydroxymethyl)piperidine-1-carbonyl)piperazin-1-yl)acetic acid ¹H NMR (500 MHz, MeOD) ¹H NMR (500 MHz, MeOD) δ 7.72 (dd, J=11.6, 2.4 Hz, 1H), 7.39 (t, J=8.5 Hz, 1H), 7.27 (ddd, J=8.8, 2.4, 1.1 Hz, 1H), 4.14 (d, J=4.7 Hz, 2H), 4.02-3.79 (m, 2H), 3.68-3.24 (m, 11H), 2.98 (dd, J=13.2, 11.4 Hz, 1H), 2.79-2.54 (m, 2H), 2.07 (d, J=14.0 Hz, 1H), 1.80 (dp, J=12.3, 3.9 Hz, 2H), 1.56-1.34 (m, 2H), 1.17 (d, J=6.2 Hz, 2H). ¹³C NMR (126 MHz, MeOD) & 172.94, 166.56, 163.12, 160.64, 160.35, 158.83, 156.89, 139.11, 139.03, 130.29, 116.07, 116.05, 114.85, 114.72, 108.07, 107.86, 64.07, 55.65, 52.00, 50.00, 43.71, 43.64, 43.30, 43.05, 38.15, 30.84, 23.99. LRMS (ESI) m/z calculated for C₂₀H₂₇ClFN₄O₅⁺ [M+H⁺] requires 457.1649; found 457.1644.

### Example 9

### General Procedure to Synthesize DL and HZ Analogs:

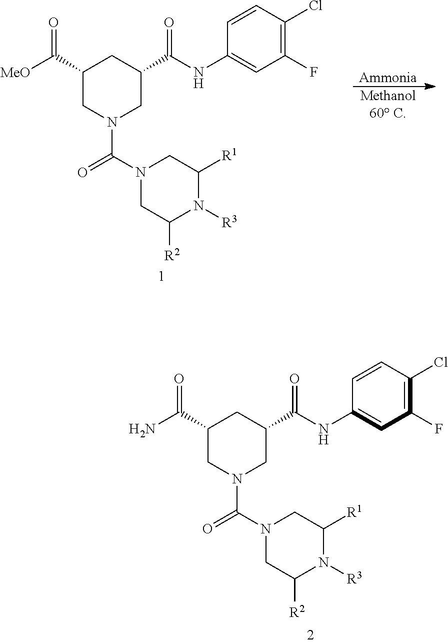

To a solution of 1 (0.4 mmol, 1.0 eq.) in methanol (0.5 M) was added 7N ammonia (210 mmol, 500 eq). The resulting mixture was heated to reflux and stirred for 16 h, then allowed to cool to room temperature and concentrated in vacuo. To the crude residue, was added DCM (dichloromethane, 5 mL) and DI water (5 mL), and the resulting mixture was extracted with DCM (3×5 mL). The organic layers were combined, washed with brine (5 mL), dried (MgSO₄), and concentrated. The resulting residue was purified by flash column chromatography to afford corresponding products.

### Example 10

DL-I-211 Characterization Data: (3S,5R)-N³-(4-chloro-3-fluorophenyl)-1-((3S,5R)-3,4,5-trimethylpiperazine-1-carbonyl)piperidine-3,5-dicarboxamide ¹H NMR (600 MHz, MeOD) δ 7.82 (m, 1H), 7.72 (br s, 1H), 7.52 (t, J=8.5 Hz, 1H), 7.35 (m, 1H), 4.09-3.85 (br m, 2H), 3.83-3.75 (br m, 2H), 3H), 3.54 (m, 2H), 2.96 (m, 2H), 2.73 (ddd, J=13.6, 10.9, 3.0 Hz, 2H), 2.69-2.44 (m, 2H), 2.33 (s, 2H), 2.29-2.10 (m, 2H), 1.96 (m, 1H), and 1.15 (m, 6H) LRMS (ESI) m/z calculated for C₂₁H₃₀ClFN₅O₃⁺ [M+H⁺] requires 454.2016; found 454.2013.

### Example 11

HZ-III-270 Characterization Data: (3S,5R)-N³-(4-chloro-3-fluorophenyl)-1-(4-methylpiperazine-1-carbonyl)piperidine-3,5-dicarboxamide ¹H NMR (600 MHz, MeOD) δ 7.83 (m, 1H), 7.72 (br s, 1H), 7.53 (t, J=8.5 Hz, 1H), 7.32 (m, 1H), 3.93-3.83 (br m, 2H), 3.82-3.73 (m, 2H), 3.66 (br s, 2H), 3.30-3.23 (m, 2H), 2.93, (m, 2H), 2.83-2.52 (m, 2H), 2.53 (d, J=4.9 Hz, 4H), 2.29 (m, 4H), and 1.83 (q, J=12.6 Hz, 1H). LRMS (ESI) m/z calculated for C₁₉H₂₆ClFN₅O₃⁺ [M+H⁺] requires 426.1703; found 426.1710.

### Example 12

HZ-IV-16 Characterization Data: (3S,5R)-1-(1H-benzo[d][1,2,3]triazole-5-carbonyl)-N³-(4-chloro-3-fluorophenyl)piperidine-3,5-dicarboxamide ¹H NMR (600 MHz, MeOD) δ 8.02 (s, 1H), 7.72 (br s, 1H), 7.95 (d, J=8.5 Hz, 1H), 7.54 (d, J=8.5 Hz, 2H), 7.45-7.09 (m, 3H), 3.88 (s, 2H), 3.01 (d, J=9.2 Hz, 2H), 2.79-2.47 (m, 4H), 2.31-2.23 (m, 1H), 2.05 (q, J=12.6 Hz, 1H). LRMS (ESI) m/z calculated for C₂₀H₁₉ClFN₆O₃⁺ [M+H⁺] requires 445.1186; found 445.1188.

### Example 13

### Biological Testing:

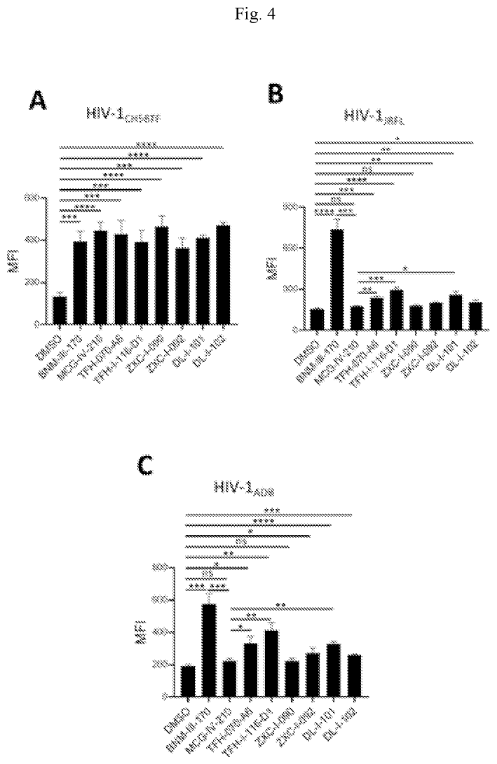

The ability of the compounds to expose the co-receptor binding site (CoRBS) were tested by measuring binding of the anti-CoRBS 17b mAb to infected cells. Briefly, primary CD4+ T cells were infected with HIV-1CH58TF, HIV-1JRFL or HIV-1AD8. Two days later, infected cells were co-incubated with 50 μM (+)-BNM-III-170, (S)-MCG-IV-210, TFH-070-A6, TFH-I-116-D1, ZXC-I-090, ZXC-I-092, DL-I-101 (ZXC-I-257), and DL-I-102 (ZXC-I-258) or the same volume of DMSO, and the 17b interaction was measured by flow cytometry after intracellular anti-p24 to identify the infected cell population. (S)-MCG-IV-210 exposed the CoRBS of cells infected with HIV-1CH58, but not the tier-2 HIV-1JRFL (FIG. 4A,B). Defined by the sensitivity to anti-body-mediated neutralization (tier-1, 2 and 3), Tier-2 viruses (HIV-1JRFL and HIV-1AD8) are representative of biologically relevant circulating HIV-1 strains which are moderately neutralized by neutralizing antibodies. Weak neutralizing antibodies, or first generation CD4mc, prevent the infection of viruses from tier-1 but not those from tier-2 or tier-3. TFH-070-A6, TFH-I-116-D1, ZXC-I-092, DL-I-101 (ZXC-I-257), and DL-I-102 (ZXC-I-258) successfully exposed the CoRBS of HIV-1JRFL infected cells, albeit to a lower level than the potent (+)-BNM-III-170 (FIG. 4B). A similar phenotype was observed when using HIV-1AD8 infected cells (FIG. 4C).

### Example 14

### (S)-MCG-IV-210 Derivatives Inhibit Viral Infection:

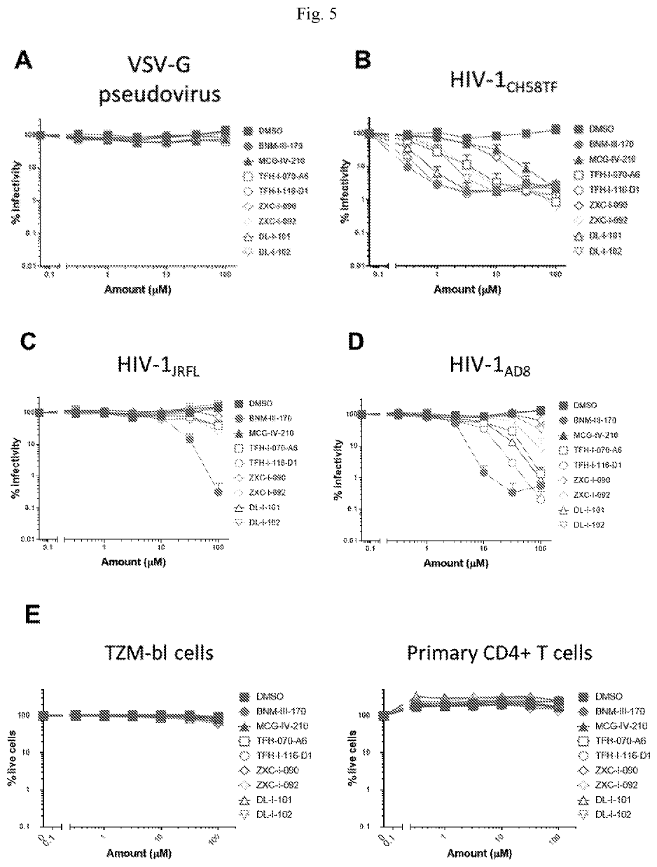

The capacity of(S)-MCG-IV-210 derivatives to neutralize viral particles with comparable infectivity of HIV-1CH58TF, HIV-1JRFL or HIV-1AD8 was evaluated by using a standard TZM-bl neutralization assay. As a positive control, (+)-BNM-III-170 was used. A VSV-G pseudovirus was used as a negative control. All tested derivatives were not toxic to TZM-bl cells (FIG. 5E, left) nor primary CD4+ T cells (FIG. 5E, right) at the tested concentrations (maximum 100 μM) and were specific to HIV-1 Env since no effect was observed with VSV-G pseudoviruses (FIG. 5A). All tested derivatives neutralized HIV-1CH58TF at low micromolar concentrations (FIG. 5A), especially TFH-I-116-D1 with an IC50 of 0.06548 μM, which were close to that of (+)-BNM-III-170 (0.03358 M) (FIG. 5A, Table 2). While HIV-1JRFL can be inhibited by small CD4-mimetic compounds, only the most potent are able to do so with (+)-BNM-III-170 having an IC50=13.49 μM. (S)-MCG-IV-210 was unable to inhibit viral infection by HIV-1JRFL (FIG. 5), but here several of the derivatives were able to do so. TFH-I-070-A6 presented an IC50 of 67.24 μM, TFH-I-116-D143.82 M and ZXC-I-092 54.22 μM (FIG. 5B, Table 2). This is the first time that analogs of(S)-MCG-IV-210 were able to neutralize HIV-1JRFL. The potency of these derivative were confirmed using another tier-2 HIV-1 strain (HIVAD8, FIG. 5C, Table 2). TFH-I-116-D1 neutralized HIV-1AD8 with an IC50 of 4.804 μM, which was similar to that of (+)-BNM-III-170 (IC50=3.749 M). Whether the H-bond formation of TFH-I-116-D1 with Env Asp368 contributed to the improved potency of this analog remains to be demonstrated.

TABLE 2
Viral neutralization. Neutralization of VSV-G HIV-
1CH58TF, HIV-1JRFL or HIV-1AD8 pseu-dovirus are
shown as IC50 (average ± SD) of at least three
independent experiments; IC50 values are in μM.

| | VSV-G | | | |
|---|---|---|---|---|
| | pseu- | | | |
| | dovirus | HIV-1_{CH58TF} | HIV-1_{JRFL} | HIV-1_{AD8} |
| DMSO | >100 | >100 | >100 | >100 |
| BNM-III-170 | >100 | 0.033 ± 0.01 | 13.49 ± 6.65 | 3.75 ± 1.3 |
| MCG-IV-210 | >100 | 3.46 ± 1.06 | >100 | >100 |
| TFH-I-070-A6 | >100 | 0.45 ± 0.09 | 67.24 ± 30 | 9.59 ± 3.4 |
| TFH-I-116-D1 | >100 | 0.065 ± 0.01 | 43.82 ± 18.6 | 4.8 ± 1.7 |
| ZXC-I-090 | >100 | 2.63 ± 0.5 | >100 | >100 |
| ZXC-I-092 | >100 | 0.75 ± 0.12 | 54.22 ± 22.8 | 34.05 ± 13.8 |
| DL-I-101 | >100 | 0.16 ± 0.03 | >100 | 10.21 ± 3.1 |
| DL-I-102 | >100 | 0.66 ± 0.25 | >100 | 87.02 ± 39 |

### Example 15

### Sensitization of HIV-1-Infected Cells to ADCC:

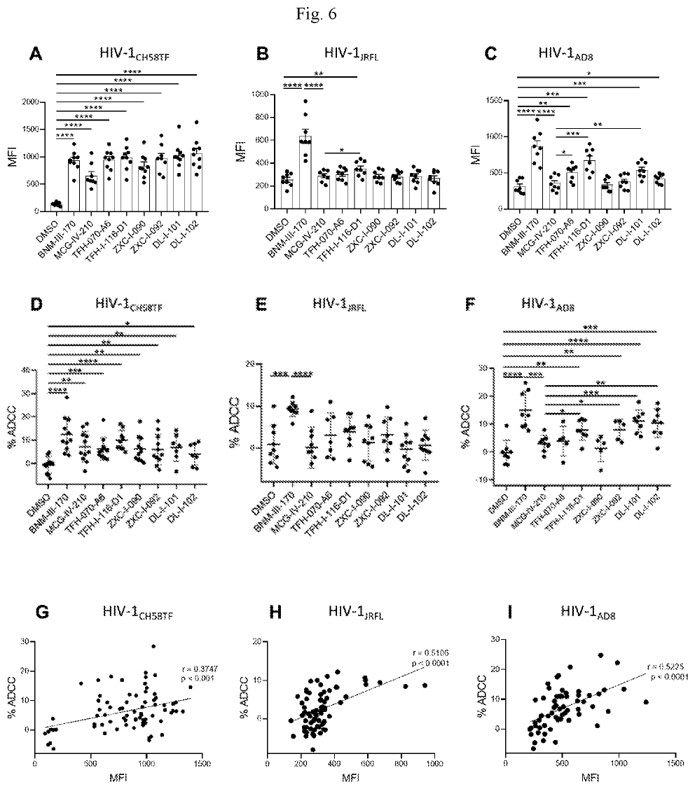

The susceptibility of primary CD4+ T cells infected with HIV-1CH58TF, HIV-1 JRFL or HIV-1AD8 to ADCC mediated was evaluated by HIV+ plasma in the absence or presence of(S)-MCG-IV-210 derivatives, using a FACS-based assay. The positive control (+)-BNM-III-170 and(S)-MCG-IV-210 enhanced the recognition of HIV-1 CH58TF infected cells and their susceptibility to ADCC mediated by HIV+ plasma. This was also the case for the new piperidine CD4mc analogs [TFH-070-A6, TFH-I-116-D1, ZXC-I-090, ZXC-I-092,DL-I-101, DL-I-102] (FIG. 6A,D). In agreement with a more CD4mc-resistant phenotype observed with HIV-1 JRFL infected cells, antibodies from PLWH recognized infected cells only in the presence of TFH-I-116-D1 but not the other derivatives (FIG. 6B); this binding was not translated into enhanced ADCC compared to what we observed with(S)-MCG-IV-210(FIG. 6E). A more heterogeneous phenotype with HIV-1_{AD8} infected cells was observed, where(S)-MCG-IV-210 did not promote HIV+ plasma binding to infected cells but TFH-070-A6, TFH-I-116-D1 or DL-I-101 did and translated into ADCC for TFH-I-116-D1 or DL-I-101 (FIG. 6C,F).

### Example 16

### BLI Competition Assay

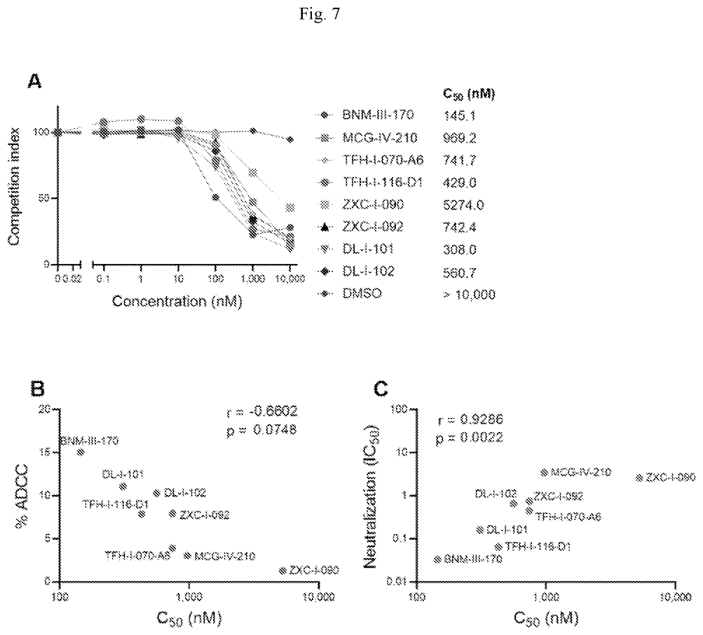

To further evaluate the potency of the different CD4mc, a biolayer interferometry (BLI) competition assay was designed. This assay measured the binding of soluble CD4 (sCD4) to the same gp120 core (LMHT gp120_{CRF01_AE} coreₑ) used for co-crystals. The CD4mc concentration required to compete out 50% of CD4-Coreₑ binding (C₅₀) is determined. FIG. 7A shows a range of C₅₀ among the different CD4mc. This measure correlates their capacity to sensitize HIV-1-infected cells to ADCC (FIG. 7B) and infection inhibition (FIG. 7C). These results are significant because BLI used the coree gp120 from a Clade A/E recombinant HIV-1 whereas the ADCC assays used cells infected with Clade B HIV-1AD8, and the neutralization assays used another Clade B HIV-1, CH58TF. Thus, suggesting that strongly conserved features of gp120 determine the vulnerabilities of HIV-1 Envs to CD4mcs.

### Materials and Methods

### Cell Lines and Isolation of Primary Cells

HEK293T human embryonic kidney cells and TZM-bl cells obtained from ATCC were grown. Primary human PBMCs, and CD4+ T cells were isolated, activated and cultured. Briefly, PBMC were obtained by leukapheresis. CD4+ T lymphocytes were then purified from resting PBMCs by negative selection using immunomagnetic beads per the manufacturer's instructions (StemCell Technologies, Vancouver, BC). CD4+ T lymphocytes were activated with phytohemagglutinin-L (10 μg/mL) for 48 h and then maintained in RPMI 1640 complete medium supplemented with rIL-2 (100 U/mL).

### Viral Production and Infection of Primary CD4+ T Cells

HIV-1 viruses were produced and titrated. Briefly, plasmids expressing the following full-length infectious molecular clones (IMCs) of HIV-1CH58TF, HIV-1JRFL or HIV-1AD8 were transfected in 293T cells by standard calcium phosphate transfection. Two days after transfection, cell supernatants were harvested, clarified by low-speed centrifugation (5 min at 1500 rpm), and concentrated by ultra-centrifugation for 1 h at 4° C. at 143,260×g over a 20% sucrose cushion. Pellets were harvested in fresh RPMI, and aliquots were stored at −80° C. until use. Viruses were then used to infect activated primary CD4+ T cells from healthy HIV-1 negative donors by spin infection at 800×g for 1 h in 96-well plates at 25° C., 48h later ˜15% of cells were infected as measured by intracellular p24 staining.

### Viral Neutralization

The viral infection assay was done. Briefly, TZM-bl target cells were seeded at a density of 1×104 cells/well in 96-well luminometer-compatible tissue culture plates (Perkin Elmer) 24 h before infection. HIV-1CH58TF, HIV-1JRFL or HIV-1AD8 viruses with comparable infectivity (˜1 million of RLU when infecting TZM-bl cells with 100 μL virus) in a final volume of 100 μL was incubated with indicated amount of different compounds or the same volume of DMSO for one hour at 37° C., then the mixture was added to the target cells followed by incubation for 48 h at 37° C.; the medium was then removed from each well, and the cells were lysed by the addition of 30 μL of passive lysis buffer (Promega) and three freeze-thaw cycles. A LB 941 TriStar luminometer (Berthold Technologies, Bad Wildbad, GermanyChennai, India) was used to measure the luciferase activity of each well after the addition of 100 μL of luciferin buffer (15 mM MgSO4, 15 mM KPO4 [pH 7.8], 1 mM ATP, and 1 mM dithio-threitol) and 50 μL of 1 mM D-luciferin potassium salt (Prolume).

### Antibodies and Plasma

The anti-CoRBS 17b mAb was used alone or in combination with different com-pounds for cell-surface staining. Plasma from different HIV-infected donors were collected, heat-inactivated and conserved. Alexa Fluor 647 conjugated Goat anti-human antibodies (Invitrogen, Waltham, MA, USA) were used as secondary Abs.

### Flow Cytometry Analysis of Cell-Surface Staining

Cell-surface staining was performed. Primary CD4 T cells were isolated from healthy donors and infected with HIV-1CH58TF, HIV-1JRFL or HIV-1AD8. Binding of HIV-1-infected cells by plasma (1:1000 dilution) or 17b mAb (5 μg/mL) in the presence or absence of 50 μM compounds was performed 48 h after infection. Cells were then incubated at 37° C. for 1 h followed by adding anti-human Alexa Fluor-647 (Invitrogen) secondary Abs for 20 min. Cells were then stained intracellularly for HIV-1 p24, using the Cytofix/Cytoperm Fixation/Permeabilization Kit (BD Biosciences, Mississauga, ON, Canada) and the fluorescent anti-p24 mAb (PE-conjugated anti-p24, clone KC57; Beckman Coulter/Immunotech). The percentage of infected cells (p24+ cells) was determined by gating the live cell population on the basis of the Aqua-Vivid viability dye staining. Samples were analyzed on an LSRII cytometer (BD Biosciences), and data analysis was performed using FlowJo vX.0.7 (Tree Star, Ashland, OR, USA).

### Cell Viability Test

To measure the potential cytotoxicity of the different CD4mcs on TZM-bl or primary CD4+ T cells, a cell viability assay using CellTiter-Glo® One Solution Assay (Promega) was performed. Briefly, TZM-bl or primary CD4+ T cells were seeded at a density of 1×104 cells/well in 96-well luminometer-compatible tissue culture plates (Perkin Elmer). After 24 h, indicated concentrations of CD4mcs up to concentrations of 100 μM were added to the cells followed by incubation for 48 h at 37° C., same volume of its vehicle, DMSO, was added as control. Then a volume of CellTiter-Glo® One Solution equal to the volume of cell culture medium present in each well was added, followed by 2 min mixing on shaker and 10min incubation at room temperature. An LB941 TriStar luminometer (Berthold Technologies) was used to measure the luciferase activity of each well.

### ADCC FACS-Based Assay

Measurement of ADCC using the FACS-based assay was performed at 48 h post-infection. Briefly, HIV-1CH58TF, HIV-1JRFL or HIV-1AD8 infected primary CD4+ T cells were stained with viability (AquaVivid; Thermo Fisher Scientific, Waltham, MA, USA) and cellular (cell proliferation dye eFluor670; eBioscience, San Diego, CA, USA) markers and used as target cells. Autologous PBMC effectors cells, stained with another cellular marker (cell proliferation dye eFluor450; eBioscience), were added at an effector: target ratio of 10:1 in 96-well V-bottom plates (Corning, Corning, NY, USA). Then the mixed cells were incubated with HIV+ plasma (1:1000), in the presence of 50 μM of compounds or with equivalent volume of vehicle (DMSO). The plates were subsequently centrifuged for 1 min at 300 g, and incubated at 37° C., 5% CO2 for 4 to 6 h before being fixed in a 2% PBS-formaldehyde solution. Samples were analyzed on an LSRII cytometer (BD Biosciences). Data analysis was performed using FlowJo vX.0.7 (Tree Star). The percentage of ADCC was calculated with the following formula: (% of p24+ cells in Targets plus Effectors)−(% of p24+ cells in Targets plus Effectors plus plasma)/(% of p24+ cells in Targets) by gating on infected lived target cells.

### CRF01_AE Core e Expression and Purification

Clade AE LM/HT and LM/HS 93TH057gp120core e protein was produced by transfection into GnTI-293F cells. Cells were grown in suspension for 7 days at 37° C. and 90% humidity. The cells were pelleted by centrifugation and the medium was filtered through a 0.2 micron filter. Protein was purified on a 17b affinity column (17b IgG co-valently linked to protein A agarose) equilibrated with phosphate buffered saline (PBS) pH 7.2. The column was washed with PBS and gp120 eluted with 0.1 M glycine pH 3.0. Eluted fractions were immediately diluted 10:1 with 1 M tris (hydroxymethyl) aminomethane-HCl (Tris-HCl) pH 8.5. Eluted protein was concentrated to approximately 10 mg/mL and the buffer was then exchanged to 50 mM sodium acetate pH 6.0 and 350 mM sodium chloride. EndoHf (New England Biolabs, Ipswich, MA, USA) was added and the sample was equilibrated overnight at 37° C. to deglycosylate the protein. Deglycosylated protein was then passed over an amylose column equilibrated in 25 mM Tris-HCl pH 7.2 and 200 mM sodium chloride to remove EndoHf (Maltose binding protein tagged EndoH). The protein was concentrated and the sample loaded onto a Superdex 200 gel filtration column (Cytiva) equilibrated in 10 mM Tris-HCl pH 7.2 and 100 mM ammonium acetate. Fractions corresponding to the de-glycosylated gp120 size were concentrated to approximately 10 mg/mL for use in crystallization trials.

### Biolayer Interferometry (BLI) Competition Assay

Biolayer interferometry (BLI) competition assay was performed on using an Octet RED96e system (ForteBio) at 25° C. with shaking at 1000 RPM. Amine Reactive Second Generation (AR2G) biosensors were hydrated in water, then activated for 300 s with a solution of 5 mM sulfo-NHS and 10 mM EDC (ForteBio) prior to amine coupling. Soluble CD4 was loaded into AR2G biosensor at 12.5 μg/mL at 25° C. in 10 mM acetate solution pH 5 (Fortebio) for 600 s then quenched into 1 M ethanolamine solution pH 8.5 (Fortebio) for 300 s. Loaded biosensor were placed in 10× kinetics buffer (ForteBio) for 120 s for baseline equilibration. Association of gp120core LMHT 100 nM (in 10X kinetics buffer) to the soluble CD4 was carried out for 180 s in the presence of various concentration of CD4mc (10 μM to 0.1 nM) or equivalent volume of DMSO prior to dissociation for 300 s. The data were baseline subtracted prior to fitting performed using a 1:1 binding model and the ForteBio data analysis software. Calculation of response was computed on all data and percentage was obtained by the following calculation: gp120core LMHT+CD4mc/gp120core LMHT×100.

Crystallization of gp120 Cores Complex with CD4mc

Crystals were grown with the hanging drop method from 10% polyethylene glycol (PEG) 3350, 5% PEG 400, and 0.1 M 4-(2-hydroxyethyl)-2-piperazineethanesulfonic acid (HEPES) pH 7.5. Crystals usually appeared within 1 to 2 weeks when incubated at 21° C. CD4 mimetic compounds were added by soaking crystals in 1 mM of the compound. Crystals were then frozen for data collection. Prior to freezing crystals were briefly soaked in the crystallization condition and compound supplemented with 20% of 2-Methyl-2,4-pentanediol (MPD) as a cryoprotectant.

Aspect 1. A compound of Formula I

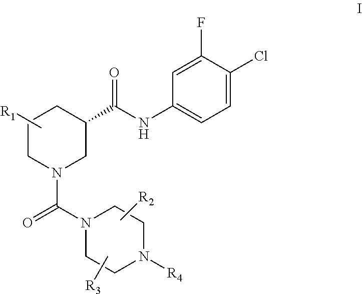

or a stereoisomer thereof, wherein:
R₁ is H or C₁₋₆alkN(R)₂, wherein each R is independently H or C₁₋₆alkyl;
R₂ is H, halo, C₀₋₆alk-aryl, substituted C₀₋₆alk-aryl, C₁₋₆alkyl, substituted C₁₋₆alkyl, or C₁₋₆haloalkyl;
R₃ is H, halo, C₀₋₆alk-aryl, substituted C₀₋₆alk-aryl, C₁₋₆alkyl, substituted C₁₋₆alkyl, or C₁₋₆haloalkyl; and
R₄ is H or C₁₋₆alkyl;
or a pharmaceutically acceptable salt thereof.

Aspect 2. The compound of Aspect 1, wherein R₁ is H.

Aspect 3. The compound of Aspect 1, wherein R₁ is —CH₂NH₂.

Aspect 4. The compound of any of the preceding Aspects, wherein R₂ is H.

Aspect 5. The compound of any one of Aspects 1 to 3, wherein R₂ is —CH₃.

Aspect 6. The compound of any one of the preceding Aspects, wherein R₃is H or —CH₃.

Aspect 7. The compound of any one of the preceding Aspects, wherein R₂is H and R₃ is H.

Aspect 8. The compound of any one of the preceding Aspects, wherein R₂is CH₃ and R₃ is —CH₃.

Aspect 9. The compound of any one of the preceding Aspects, wherein R₄is —CH₃.

Aspect 10. The compound of any one of the preceding Aspects, that is a compound of Formula IA, or a stereoisomer thereof or a pharmaceutically acceptable salt thereof:

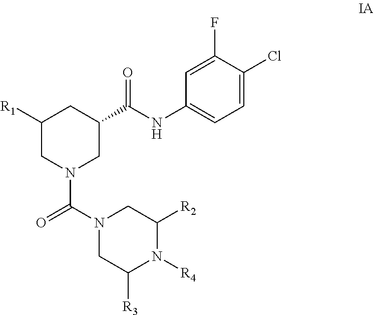

Aspect 11. The compound of any one of the preceding Aspects that is:

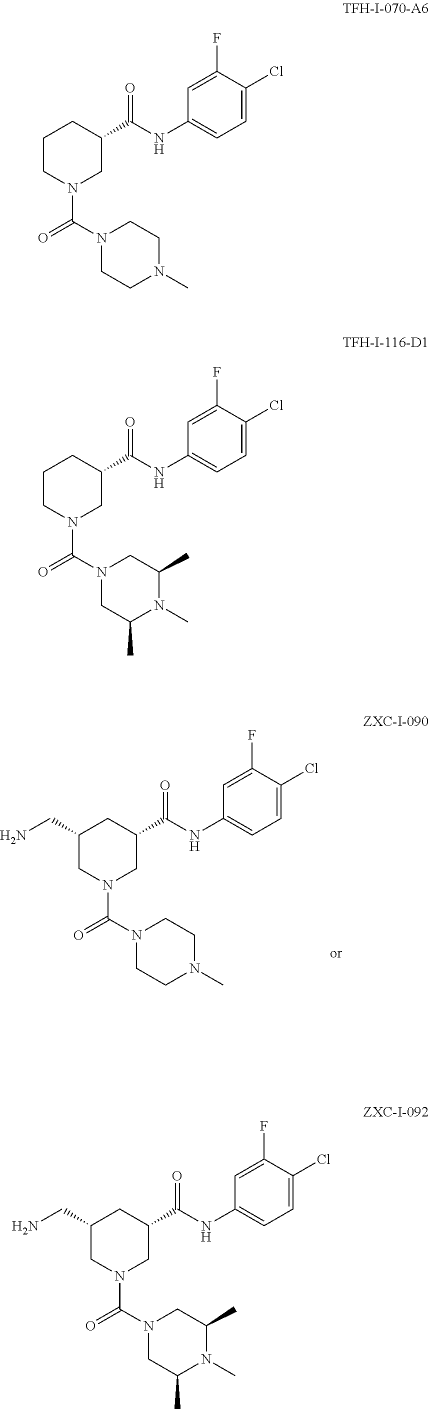 [SMILES: CN1CCN(C(=O)N2CCC[C@H](C(=O)Nc3ccc(Cl)c(F)c3)C2)CC1 ; C[C@@H]1CN(C(=O)N2CCC[C@H](C(=O)Nc3ccc(Cl)c(F)c3)C2)C[C@H](C)N1C ; CN1CCN(C(=O)N2C[C@H](CN)C[C@H](C(=O)Nc3ccc(Cl)c(F)c3)C2)CC1 ; C[C@@H]1CN(C(=O)N2C[C@H](CN)C[C@H](C(=O)Nc3ccc(Cl)c(F)c3)C2)C[C@H](C)N1C]

Aspect 12. A pharmaceutical composition comprising a compound of any one of the preceding Aspects and a pharmaceutically acceptable excipient.

Aspect 13. A method of treating or preventing HIV-1 replication in a mammal comprising administering to the mammal in need thereof a compound of any one of Aspects 1-11 or a pharmaceutical composition of Aspect 12.

Aspect 14. A method of preventing the transmission of HIV-1 to a mammal comprising administering to the mammal a compound of any one of Aspects 1-11 or a pharmaceutical composition of Aspect 12.

Aspect 15. The method of any one of Aspects 13 or 14, further comprising administering to the mammal one or more antiretrovirals.

Aspect 16. A compound of Formula I

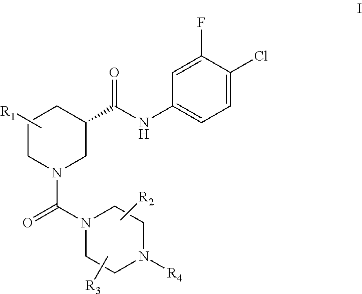

or a stereoisomer thereof, wherein:
R₁ is H, C₁₋₆alkN(R)₂, or C₁₋₆alkOR′, wherein each R is independently H or C₁₋₆alkyl;
R′ is independent H or C₁₋₆alkyl.
R₂ is H, halo, C₀₋₆alk-aryl, substituted C₀₋₆alk-aryl, C₁₋₆alkyl, substituted C₁₋₆alkyl, or C₁₋₆haloalkyl;
R₃ is H, halo, C₀₋₆alk-aryl, substituted C₀₋₆alk-aryl, C₁₋₆alkyl, substituted C₁₋₆alkyl, or C₁₋₆haloalkyl; and
R₄ is H, C₁₋₆alkyl, C₁₋₆alkCOOH, or C(O)C₁₋₆alkN(R″)₂, wherein R″ is independent H or C₁₋₆alkyl.
or a pharmaceutically acceptable salt thereof.

Aspect 17. The compound of Aspect 16, wherein R₁ is H.

Aspect 18. The compound of Aspect 16, wherein R₁ is —CH₂NH₂.

Aspect 19. The compound of Aspect 16, wherein R₁ is —CH₂OH.

Aspect 20. The compound of Aspect 16, wherein R₁ is —CH₂CH₂CH₂OH.

Aspect 21. The compound of any of the preceding Aspects, wherein R₂ is H.

Aspect 22. The compound of any one of Aspects 16 to 20, wherein R₂ is —CH₃.

Aspect 23. The compound of any one of the preceding Aspects, wherein R₃ is H or —CH₃.

Aspect 24. The compound of any one of the preceding Aspects, wherein R₂ is H and R₃ is H.

Aspect 25. The compound of any one of the preceding Aspects, wherein R₂ is CH₃ and R₃ is —CH₃.

Aspect 26. The compound of any one of the preceding Aspects, wherein R₄ is —CH₃.

Aspect 27. The compound of any one of Aspects 16 to 25, wherein R₄ is —CH₂COOH.

Aspect 28. The compound of any one of Aspects 16 to 25, wherein R₄ is —C(O)CH₂NH₂.

Aspect 29. The compound of any one of the preceding Aspects, that is a compound of Formula IA, or a stereoisomer thereof or a pharmaceutically acceptable salt thereof:

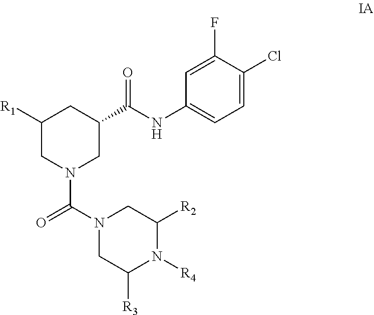

Aspect 30. The compound of any one of the preceding Aspects that is:

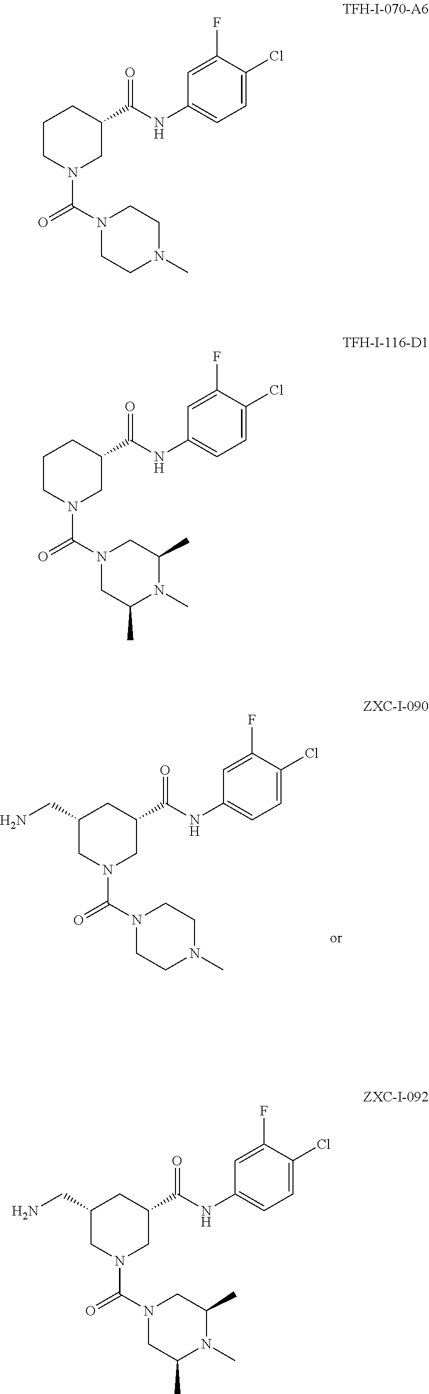 [SMILES: CN1CCN(C(=O)N2CCC[C@H](C(=O)Nc3ccc(Cl)c(F)c3)C2)CC1 ; C[C@@H]1CN(C(=O)N2CCC[C@H](C(=O)Nc3ccc(Cl)c(F)c3)C2)C[C@H](C)N1C ; CN1CCN(C(=O)N2C[C@H](CN)C[C@H](C(=O)Nc3ccc(Cl)c(F)c3)C2)CC1 ; C[C@@H]1CN(C(=O)N2C[C@H](CN)C[C@H](C(=O)Nc3ccc(Cl)c(F)c3)C2)C[C@H](C)N1C]

Aspect 31. The compound of any one of the preceding Aspects that is:

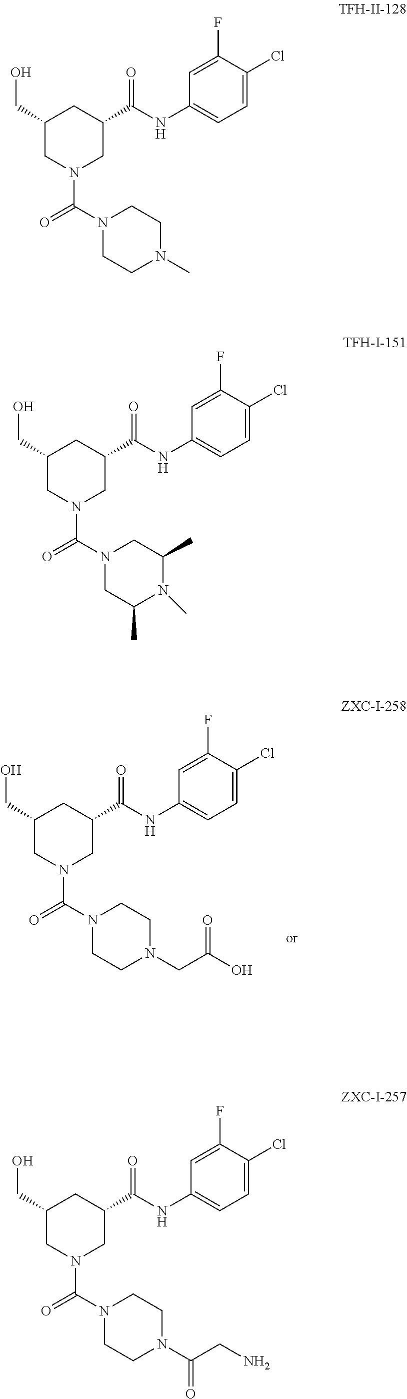 [SMILES: CN1CCN(C(=O)N2C[C@H](CO)C[C@H](C(=O)Nc3ccc(Cl)c(F)c3)C2)CC1 ; C[C@@H]1CN(C(=O)N2C[C@H](CO)C[C@H](C(=O)Nc3ccc(Cl)c(F)c3)C2)C[C@H](C)N1C ; O=C(O)CN1CCN(C(=O)N2C[C@H](CO)C[C@H](C(=O)Nc3ccc(Cl)c(F)c3)C2)CC1 ; NCC(=O)N1CCN(C(=O)N2C[C@H](CO)C[C@H](C(=O)Nc3ccc(Cl)c(F)c3)C2)CC1]

Aspect 32. A pharmaceutical composition comprising a compound of any one of the preceding Aspects and a pharmaceutically acceptable excipient.

Aspect 33. A method of treating or preventing HIV-1 replication in a mammal comprising administering to the mammal in need thereof a compound of any one of Aspects 16-31 or a pharmaceutical composition of Aspect 32.

Aspect 34. A method of preventing the transmission of HIV-1 to a mammal comprising administering to the mammal a compound of any one of Aspects 16-31 or a pharmaceutical composition of Aspect 32.

Aspect 35. The method of any one of Aspects 32 or 33, further comprising administering to the mammal one or more antiretrovirals.

Nothing in this specification should be considered as limiting the scope of this disclosure. All examples presented are representative and non-limiting. The above-described embodiments can be modified or varied, as appreciated by those skilled in the art in light of the above teachings. It is therefore to be understood that, within the scope of the claims and their equivalents, the embodiments disclosed herein can be practiced otherwise than as specifically described.

1. A compound of Formula II: 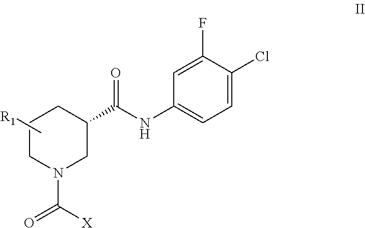
or a stereoisomer thereof, wherein:
R₁ is H, C₁₋₆alkN(R)₂, C₁₋₆alkOR′, or C₁₋₆alk(O)N(R)₂, wherein each R is, independently, H or C₁₋₆alkyl; R′ is independent H or C₁₋₆alkyl.
X is 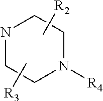
or a bicyclic ring system; wherein the bicyclic ring system is a 9 membered bicyclic aromatic ring system consisting of a 6 membered ring fused with a 5-membered ring; wherein the 5-membered ring comprises one, two or three heteroatoms that are each, independently, O, S, or N;
the bicyclic aromatic ring system is attached to the remainder of the molecule via a ring carbon atom of the 5- or 6-membered ring or a ring nitrogen atom of the 5-membered ring; the bicyclic ring system is optionally substituted on the carbon atoms with one, two, three, or four substituents each independently halo, oxo, —OH, —NH₂, —NH-C₁₋₄alkyl, C₂₋₆alkenyl, or C₁₋₄alkyl,
R₂ is H, halo, C₀₋₆alk-aryl, substituted C₀₋₆alk-aryl, C₁₋₆alkyl, substituted C₁₋₆alkyl, or C₁₋₆haloalkyl;
R₃ is H, halo, C₀₋₆alk-aryl, substituted C₀₋₆alk-aryl, C₁₋₆alkyl, substituted C₁₋₆alkyl, or C₁₋₆haloalkyl; and
R₄ is H, C₁₋₆alkyl, C₁₋₆alkCOOH, or C(O)C₁₋₆alkN(R″)₂, wherein R″ is independently H or C₁₋₆alkyl,
or a pharmaceutically acceptable salt thereof.

2. The compound of claim 1, wherein X is

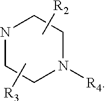

3. The compound of claim 1, wherein X is the bicyclic ring system.

4. The compound of claim 3, wherein X is

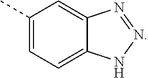

5. The compound of claim 1, wherein R₁, R₂, R₃, and/or R₄ is H.

6. The compound of claim 1, wherein R₁ is —CH₂NH₂.

7. The compound of claim 1, wherein R₁ is —CH₂OH or —CH₂CH₂CH₂OH.

8. (canceled)

9. The compound of claim 1, wherein R₁ is —C(O)—NH₂.

10. (canceled)

11. The compound of claim 1, wherein R₂, R₃, and/or R₄ is —CH₃.

12. (canceled)

13. The compound of claim 1, wherein R₂ and R₃ are H or R₂ and R₃ are CH₃.

14-15. (canceled)

16. The compound of claim 1, wherein R₄ is —CH₂COOH.

17. The compound of claim 1, wherein R₄ is —C(O)CH₂NH₂.

18. The compound of claim 1, that is a compound of Formula IA, or a stereoisomer thereof or a pharmaceutically acceptable salt thereof:

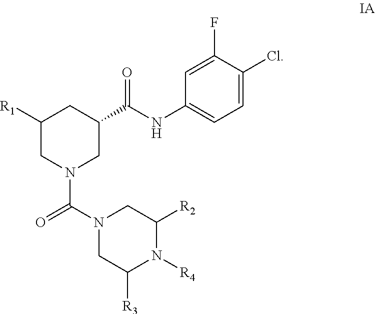

19. The compound of claim 1 that is:

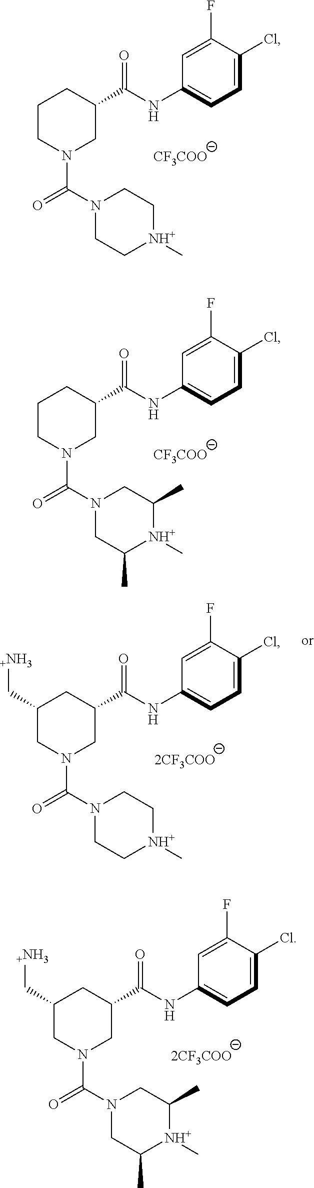

20. The compound of claim 1 that is:

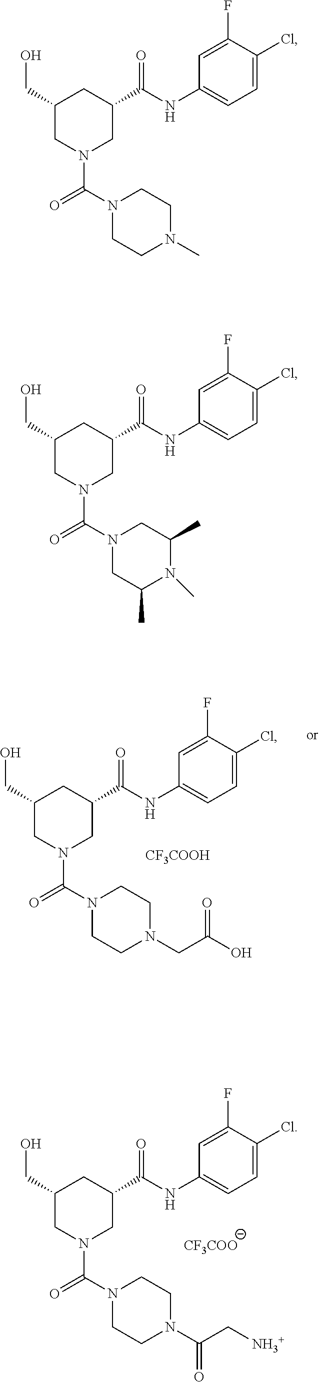

21. The compound of claim 1 that is:

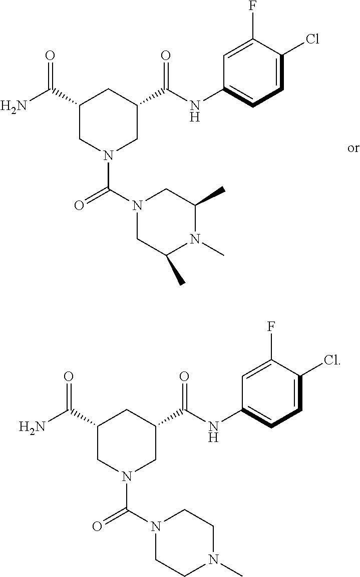

22. The compound of claim 1 that is:

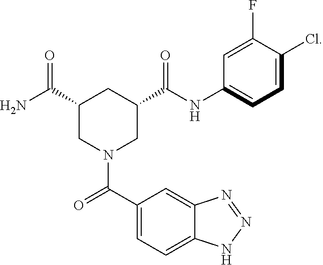

23. A pharmaceutical composition comprising a compound of claim 1 and a pharmaceutically acceptable excipient.

24. A method of treating or preventing HIV-1 replication in a mammal or preventing the transmission of HIV-1 to a mammal, comprising administering to the mammal in need thereof a compound of claim 1.

25. (canceled)

26. The method of claim 24, further comprising administering to the mammal one or more antiretrovirals.
